(19) United States
(12) Patent Application Publication
(10) Pub. No.: US 2025/0262156 A1
(43) Pub. Date: Aug. 21, 2025

(54) COMPOSITIONS OF KINETIC NANOPARTICLES CONTAINING NUCLEIC ACIDS, POLYCATIONS, AND LIPIDS WITH DEFINED SIZES, AND METHOD OF PRODUCING THE SAME

(71) Applicant: THE JOHNS HOPKINS UNIVERSITY, Baltimore, MD (US)

(72) Inventors: Hai-Quan MAO, Baltimore, MD (US); Stephany YI TZENG, Baltimore, MD (US); Jordan J. GREEN, Woodstock, MD (US); Yizong HU, Baltimore, MD (US); Sashank REDDY, Baltimore, MD (US)

(21) Appl. No.: 18/859,372

(22) PCT Filed: Apr. 26, 2023
(86) PCT No.: PCT/US2023/019991

§ 371 Date: Oct. 23, 2024

Related U.S. Application Data
(60) Provisional application No. 63/335,089, filed on Apr. 26, 2022.

(51) Int. Cl. A61K 9/1273; A61K 9/00; A61K 9/1277; A61K 9/51; A61K 47/26; A61K 48/00; B82Y 5/00; C12N 15/88

(52) U.S. Cl. CPC A61K 9/1273; A61K 9/0019; A61K 9/1277; A61K 9/5123; A61K 9/5146; A61K 47/26; A61K 48/0041; A61K 48/0075; C12N 15/88; B82Y 5/00

(57) ABSTRACT

Hybrid nanoparticles having a defined size in a range between about 50 nm to about 1000 nm prepared by a kinetic assembly process are disclosed. The hybrid nanoparticles comprise a biodegradable polycation and a PEGylated lipid and include a nucleic acid including, but not limited to, plasmid DNA, messenger RNA (mRNA), small interfering RNA (siRNA), and the like. The assembled hybrid nanoparticles can be used for gene delivery therapy in vivo through various delivery routes and ex vivo and in vitro to transfect cells of interest. The disclosed hybrid nanoparticles with certain sizes within a sub-micron range exhibited significantly improved transfection efficiency compared to nanoparticles without size control.

### STATEMENT OF GOVERNMENTAL INTEREST

This invention was made with government support under grant AI160738 and EB028239 awarded by the National Institutes of Health. The government has certain rights in the invention.

### BACKGROUND

Gene therapy has become an increasingly valuable modality for treating congenital and acquired conditions, and prophylactic and treatment vaccines. Many current gene therapies include the use of vectorized viruses based on lentivirus (LVVs) and adeno-associated virus (AAVs). Benchmark transfection vehicles include calcium phosphate, lipofectamine, and poly(ethylenimine) (PEI).

In a typical transfection procedure using PEI, pDNA mixture and PEI are separately dissolved in a serum-reduced medium. Following manual mixing, the suspension is typically incubated for between 0-60 min to allow for complete polyelectrolyte complex (PEC) coacervation, after which it is added to the cultures. The pDNA/PEI particles facilitate cell entry, endosomal escape, and nuclear transport of pDNAs, resulting in transcription of the viral RNAs, as well as expression of packaging and envelope proteins. Successful co-transfection of all species of pDNAs is required to yield functional LVVs. Additionally, scalable and reproducible production methods are essential to ensure consistent quality of LVVs and safe, efficacious therapeutic outcomes. Such quality production is only possible when the transfection process is fully controlled to yield high degrees of efficiency and consistency.

The widely adopted method to manually prepare pDNA/PEI particles immediately before transfection, however, suffers from high batch-to-batch variation, negatively affecting the reliability and efficiency of viral vector production. Such inconsistencies readily occur due to several factors including: (1) complexity associated with assembly of particles composed of multiple pDNAs of different lengths; (2) difficulty in achieving uniform mixing throughout the mixing vessel (spatial heterogeneity); (3) difficulty in maintaining a consistent pDNA/PEI ratio during the sequential addition processes (temporal heterogeneity); and (4) varied incubation times of particles formed throughout the production process. More importantly, such a manual preparation process is prone to operator-dependent variability and is challenging to scale up. For example, LVV production at pharmaceutical batch sizes of hundreds of liters requires liter-scale mixing of pDNA and PEI solutions, raising challenges of mass transfer in liquid handling. Therefore, it is critical to develop an engineering approach to produce shelf-stable pDNA/PEI particles in a highly scalable and consistent fashion to ensure high transfection efficiency with ease-of-use features.

A flash nanocomplexation (FNC) technique for scalable production of pDNA/PEI nanoparticles was recently developed. Discrete sub-100 nm nanoparticles have been successfully generated in a lyophilized form for systemic delivery applications in vivo. These small nanoparticles, however, are sub-optimal for in vitro transfection in viral vector production cell lines (i.e., HEK293T or HEK293F cells), showing only a fraction of the peak transfection efficiency of the particles obtained by a standard manual mixing method. Size-dependent transfection efficiency for particles of sizes beyond 100 nm, however, has rarely been previously reported, and little mechanistic understanding exists. The poor insight into size-dependent transfection efficiency of pDNA/PEI particles reflects the lack of methods to control the size and stability of these particles in the range of 200 nm to 1000 nm. Conventional pipette mixing or dropwise addition without control of assembly kinetics results in particles with unpredictable sizes and a high degree of instability.

### SUMMARY

In some aspects, the presently disclosed subject matter provides a method for preparing a plurality of hybrid nanoparticles having a defined size, the method comprising:
(a) mixing a biodegradable cationic polymer and one or more nucleic acids to form a first plurality of charge-neutralized complexes;
(b) disposing the plurality of charge-neutralized complexes in a buffer solution for a period of time to induce particle growth to form a second plurality of charge-neutralized complexes, wherein the second plurality of charge-neutralized complexes has a particle size greater than a particle size of the first plurality charge-neutralized complexes; and
(c) adding a hydrophobic PEGylated lipid to the second plurality of charge-neutralized complexes to quench particle growth and to form a plurality of hybrid nanoparticles having a defined size.

In some aspects, the first plurality of charge-neutralized complexes are formed under low salt concentration and low pH conditions. The low salt concentration ranges from an ionic strength equivalent to about 1 mM to about 40 mM NaCl, while the low pH ranges from about 2 to about 5.5.

In some aspects, the second plurality of charge-neutralized complexes are formed under high salt concentration and high pH conditions in the presence of one or more multivalent ions. In certain aspects, the one or more multivalent ions comprise a negatively-charged ion selected from phosphate, citrate, EDTA, pyrophosphate, ATP, tripolyphosphate, and hexametaphosphate. In certain aspects, the one or more multivalent ions comprise a positively-charged magnesium, calcium, ferrous, and aluminum. In certain aspects, the high salt concentration ranges from an ionic strength equivalent to about 40 mM to about 300 mM NaCl, while the high pH ranges from about 5.5 to about 9.0.

In some aspects, the biodegradable cationic polymer and the one or more nucleic acids are mixed through pipetting, at a T junction flow path, in a microfluidic channel or mixer, in a multi-inlet vortex mixer, or in a confined impinging jet (CIJ) mixer. In particular aspects, the biodegradable cationic polymer and the one or more nucleic acids are mixed in a confined impinging jet (CIJ) mixer.

In certain aspects, the biodegradable cationic polymer and the one or more nucleic acids are mixed at a pH of about 5.

In certain aspects, the biodegradable cationic polymer and the one or more nucleic acids are mixed at a ratio of about 3:1 nucleic acid:cationic polymer.

In certain aspects, the salt concentration in the first plurality of charge-neutralized complexes is equivalent to 10 mM NaCl.

In certain aspects, the buffered solution comprises phosphate buffered saline (PBS).

In certain aspects, the period of time the first plurality of charge-neutralized complexes is disposed in the buffer solution has a range from about 60 milliseconds to about 300 min. In particular aspects, the period of time the first plurality of charge-neutralized complexes is disposed in the buffer solution has a range selected from about 0.001 min to about 30 min; from about 0.001 min to about 20 min; about 0.001 min to about 19 min; about 0.001 min to about 18 min; about 0.001 min to about 17 min; about 0.001 min to about 16 min; about 0.001 min to about 15 min; about 0.001 min to about 14 min; about 0.001 min to about 13 min; about 0.001 min to about 12 min; about 0.001 min to about 11 min; about 0.001 min to about 10 min; about 0.01 min to about 9 min; about 0.01 min to about 8 min; about 0.01 min to about 7 min; about 0.01 min to about 6 min; about 0.01 min to about 5 min; about 0.01 min to about 4 min; about 0.01 min to about 3 min; about 0.01 min to about 2 min; and about 0.01 min to about 1 min.

In certain aspects, the salt concentration in the second plurality of charge-neutralized complexes is equivalent to 150 mM NaCl.

In certain aspects, the pH in the second plurality of charge-neutralized complexes is 7.4.

In some aspects, the PEGylated lipid comprises 1,2-dimyristoyl-sn-glycero-3-methoxypolyethylene glycol (DMG-PEG). In particular aspects, the PEGylated lipid is DMG-PEG2000. In more particular aspects, the DMG-PEG2000 comprises greater than about 5% of a mass concentration of the hybrid nanoparticle. In yet more particular aspects, the DMG-PEG2000 comprises between about 5% to about 20% of the mass concentration of the hybrid nanoparticle.

In some aspects, the PEGylated lipid further comprises a chemically-active moiety. In certain aspects, the method further comprises functionalizing the chemically-active moiety with a targeting ligand or other biologically active chemical structures.

In some aspects, the one or more nucleic acids is selected from an antisense oligonucleotide, cDNA, genomic DNA, guide RNA, plasmid DNA (pDNA), including a mixture of different species of pDNA, vector DNA, mRNA, miRNA, piRNA, shRNA, and siRNA. In particular aspects, the one or more nucleic acids comprises mRNA. In more particular aspects, the plurality of hybrid nanoparticles comprises between about 2 to about 1500 copies of mRNA per particle.

In some aspects, the plurality of hybrid nanoparticles have an average particle size having a range from about 50 nm to about 1000 nm; about 50 nm to about 900 nm; about 50 nm to about 800 nm; about 50 nm to about 700 nm; about 50 nm to about 600 nm; about 50 nm to about 500 nm; about 50 nm to about 400 nm; about 50 nm to about 300 nm; about 50 nm to about 200 nm; and about 50 nm to about 100 nm. In particular aspects, the plurality of hybrid nanoparticles have an average particle size of about 400 nm.

In some aspects, the plurality of hybrid nanoparticles have a zeta-potential of between about 2 mV and about 6 mV.

In some aspects, the plurality of hybrid nanoparticles have an encapsulation efficiency of between about 80% to about 100%.

In some aspects, the biodegradable cationic polymer comprises a poly(beta-amino ester) (PBAE). In certain aspects, the PBAE comprises a compound of formula (I):

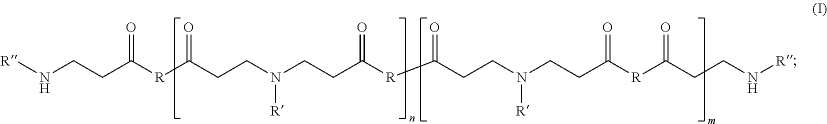

wherein:
m and n are each independently an integer from 1 to 10,000;
R comprises a divalent radical comprising a biodegradable ester linkage and/or a bioreducible disulfide linkage;
each R′ can be the same or different and is selected from a hydrophobic sidechain or a hydrophilic sidechain comprising a monovalent radical derived from an amine monomer;
R″ is monovalent radical derived from an amine-containing end capping group; and pharmaceutically acceptable salts thereof.

In particular aspects, each R is:

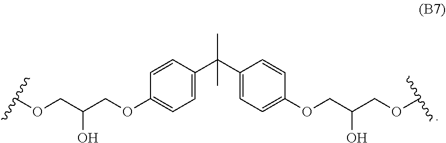 [SMILES: *OCC(O)COc1ccc(C(C)(C)c2ccc(OCC(O)CO*)cc2)cc1]

In some aspects, at least one R′ comprises:

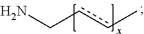

wherein x is an integer selected from 0, 1, 2, 3, 4, 5, 6, 7, 8, 9, 10, 11, 12, 13, 14, 15, 16, 17, 18, 19, and 20; and wherein

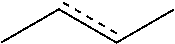

can De a single or double pond in one or more x repeating units. In certain aspects, at least one R′ is selected from:

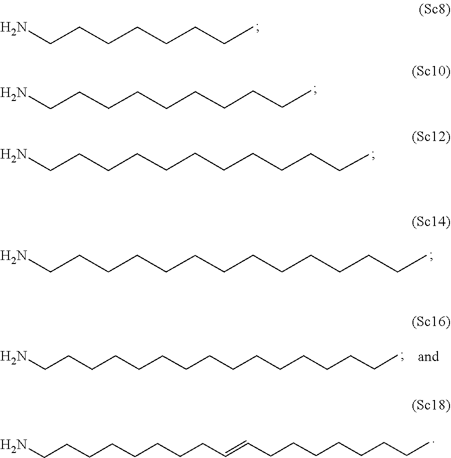

In particular aspects, at least one R′ is:

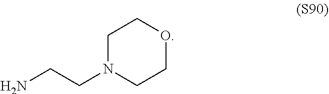

In particular aspects, R″ is:

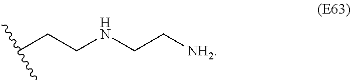 [SMILES: *CCNCCN]

In yet more particular aspects, the compound of formula (I) is:

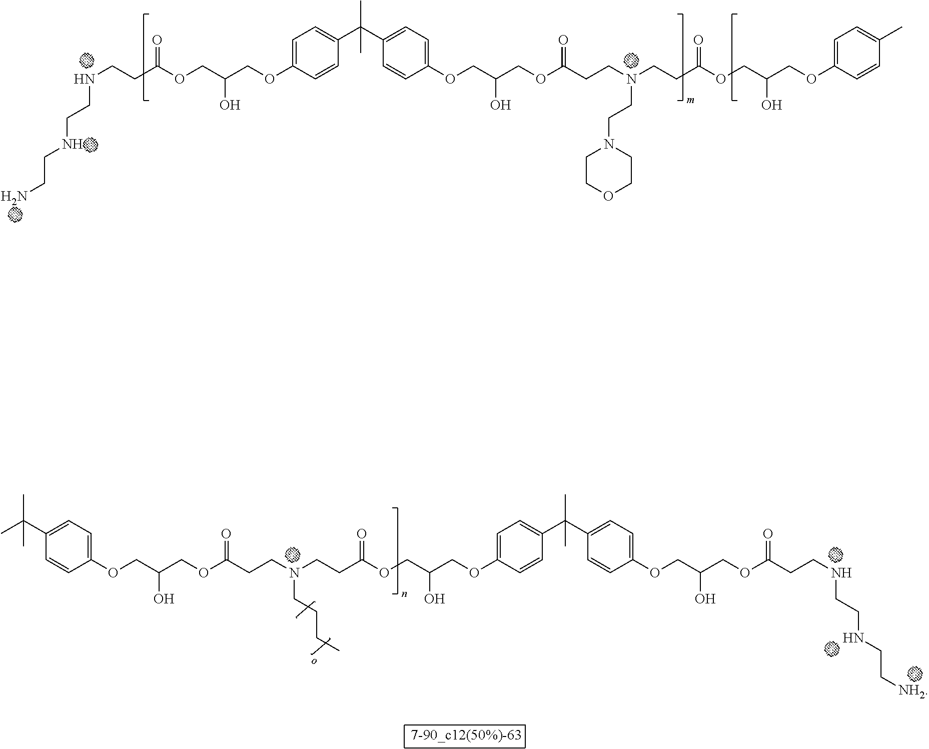

In some aspects, the method further comprises an excipient for cryo-preservation of the plurality of particles. In certain aspects, the excipient is selected from a saccharide and a sugar alcohol. In particular aspects, the saccharide is selected from a monosaccharide, a disaccharide, and a polysaccharide. In particular aspects, the sugar alcohol is selected from arabinose, glucose, fructose, ribose, mannose, sucrose, trehalose, lactose, maltose, starches, dextran, mannitol, and sorbitol. In more particular aspects, the sugar alcohol is 15% w/v trehalose.

In some aspects, the presently disclosed subject matter provides a hybrid nanoparticle comprising a PBAE, a nucleic acid, and a PEGylated lipid, wherein nucleic acid comprises mRNA, the PEGylated lipid comprises DMG-PEG2000, and the PBAE comprises a compound of the following formula:

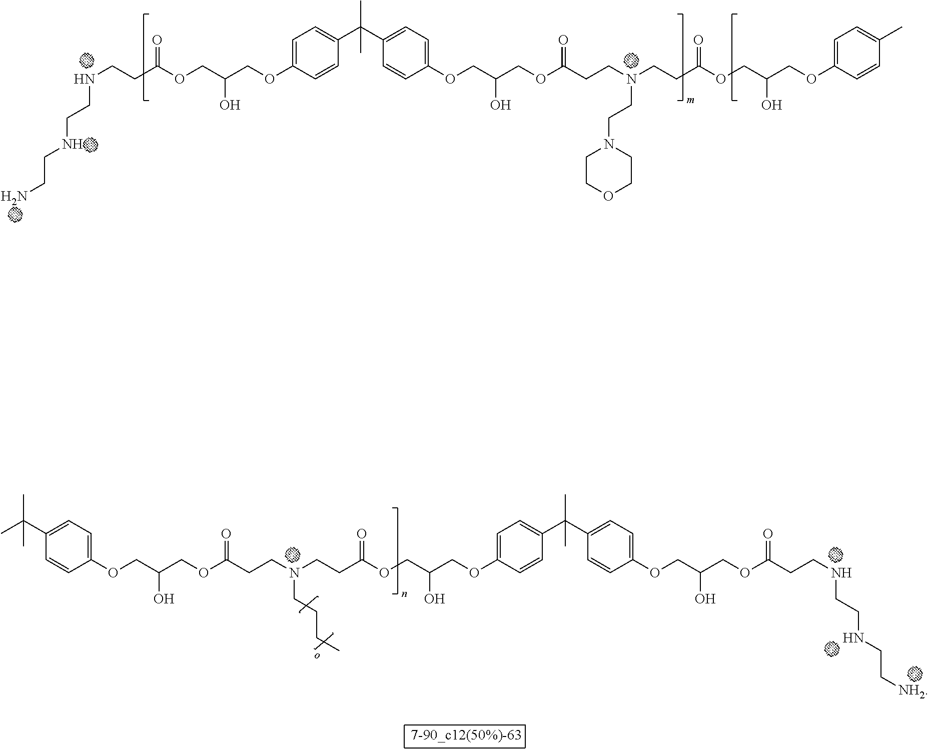

In certain aspects, the hybrid nanoparticle has particle size of about 400 nm. In certain aspects, the DMG-PEG2000 comprises between about 5% to about 20% of a mass concentration of the nanoparticle. In certain aspects, the hybrid nanoparticle comprises between about 2 to about 1500 copies of mRNA per particle. In certain aspects, the plurality of hybrid nanoparticles have a zeta-potential of between about 2 mV and about 6 mV. In certain aspects, the hybrid nanoparticle has an encapsulation efficiency of between about 80% to about 100%. In certain aspects, the PEGylated lipid further comprises a chemically-active moiety. In certain aspects, the chemically-active moiety is functionalized with a targeting ligand or other biologically active chemical structure.

In some aspects, the presently disclosed subject matter provides a method for transfecting a cell, the method comprising administering to the cell a presently disclosed hybrid nanoparticle. In certain aspects, the method comprises ex vivo or in vitro transfection and gene editing.

In some aspects, the presently disclosed subject matter provides a method for delivering mRNA to a tissue, the method comprising administering to the tissue a presently disclosed hybrid nanoparticle. In certain aspects, the tissue is selected from lung, liver, kidney, heart, and spleen.

In some aspects, the presently disclosed subject matter provides a method for delivering a gene to a subject, the method comprising administering a hybrid nanoparticle to the subject. In certain aspects, the administration is in vivo. In particular aspects, the method comprises intravenous administration.

In some aspects, the presently disclosed subject matter provides a method for treating a disease, condition, or disorder, the method comprising administering to a subject in need of treatment thereof, a presently disclosed hybrid nanoparticle. In certain aspects, the disease, condition, or disorder comprises cancer.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B are schematic representations showing supra-molecular assembly using mRNA and poly(beta-amino ester) (PBAE). FIG. 1A shows a representative poly(beta-amino acid) (PBAE) structure suitable for use with the presently disclosed methods. Without wishing to be bound to any one particular theory, it is thought that the hydrophobic side chain (e.g., at least one R′ is hydrophobic) plays an essential role in size control; and FIG. 1B is a scheme of the presently disclosed method for controlling size;

FIG. 2A, FIG. 2B, FIG. 2C, FIG. 2D, and FIG. 2E show size control of mRNA/PBAE nanoparticles from 100 to 1000 nm. FIG. 2A demonstrates that the size growth curve as a result of challenging the building block mRNA/PBAE nanoparticles with PBS, and the stabilization at desired sizes of 100, 200, 300, 400, 550, 700, and 1000 nm by addition of DMG-PEG2000; FIG. 2B. shows the encapsulation efficiency (percentage of mRNA molecules that are complexed with PBAE as a result of the presently disclosed size control method, and the surface charge (zeta-potential) of the assembled particles. As a result of insertion of PEG onto the surfaces of the particles, the PEG molecules (hydrophilic, neutral) efficiently screen the original positive charges on PBAE molecules; FIG. 2C shows the effect of dose of DMG-PEG on the surface charge and stabilization effect of the particles. While all PEG ratios resulted in efficient charge screening, a minimum of PEG>5% was required to effectively stop the size growth; FIG. 2D shows the size distributions (assessed by dynamic light scattering) of assembled particles; and FIG. 2E shows the payload capacities (mRNA copies per particle) of assembled particles (assessed by cylindrical illumination confocal microscopy technique);

FIG. 3A, FIG. 3B, FIG. 3C, FIG. 3D, and FIG. 3E show the transfection of bone marrow-derived macrophages (BMDMs). The cells were either treated by Cy5-mRNA loaded particles to assess cellular uptake at 4 h post-dosage or treated by GFP-mRNA loaded particles to assess single-cell transfection profile at 24 h post-dosage. The viability assay was conducted using alamarBlue kit, assessed at 24 h post-dosage. FIG. 3A shows the effect of particle size and dose of mRNA/PBAE particles on the cellular uptake by BMDMs; FIG. 3B is, with a dose of 0.4 μg/mL, the cellular uptake profiles of the particles; FIG. 3C shows the effect of particle size and dose of mRNA/PBAE particles on viability of BMDMs; FIG. 3D shows the effect of particle size and dose of mRNA/PBAE particles on the percentage transfected (GFP+ BMDMs); and FIG. 3E shows the effect of particle size and dose of mRNA/PBAE particles on the single-cell transfection level (mean fluorescent intensity, or MFI of GFP+ BMDMs);

FIG. 4A, FIG. 4B, FIG. 4C, and FIG. 4D demonstrate the transfection benefit from size-controlled mRNA/PBAE nanoparticles. Each animal was intravenously injected particles loaded with luciferase mRNA at different sizes. At 12-h post-injection, luciferin substrate was injected to assess the luciferase expression level in vivo. The lungs, liver and spleen were then harvested to be imaged ex vivo. The organs were then homogenized by lysis buffer to assess the absolute luciferase concentration locally. FIG. 4A shows the live-animal imaging or ex-vivo imaging results assessing luciferase expression; FIG. 4B shows the absolute local luciferase concentration within each organ; FIG. 4C shows the kinetics of luciferase signal examined from live-animal imaging over a period of 3 days; and FIG. 4D demonstrates that each animal was implanted a CT26 tumor and particles were injected into the implanted tumor. At 24-h post-injection, luciferin substrate was injected to assess the luciferase expression level in vivo;

FIG. 5 and FIG. 6 illustrates that an Ai-9 mouse model was used to assess the cell types transfected by mRNA/PBAE particles at different sizes. Top panel: The data was normalized to each individual cell type. For example, a number of 30% for cell type A means that out of every 100 A cells, there were 30 of them successfully transfected. Bottom panel: The data was normalized to all cells transfected. The number of circles indicate the transfection level (i.e., 400-nm group had the highest transfection efficiency). For example, the 33 gray circles (representing B cells) for 400-nm particles-treated liver mean that out of every 100 transfected cells, 33 of them were B cells;

FIG. 7A, FIG. 7B, and FIG. 7C show: (FIG. 7A) Cellular uptake of PBAE/Cy5-mRNA kinetic nanoparticles (KNPs) by BMDMs; (FIG. 7B) Transfection efficiency in BMDMs by PBAE/GFP-mRNA KNPs; and (FIG. 7C) Transfection efficiency in macrophages in different organs following i.v. injection of PBAE/mRNA KNPs with different sizes;

FIG. 8A, FIG. 8B, and FIG. 8C show: (FIG. 8A) Detection of F4/80⁻/CD11b⁺ homing monocytes following i.v. injection of 400-nm KNPs; (FIG. 8B) High transfection efficiency in the homed monocytes by 400-nm KNPs; and (FIG. 8C) Size-dependent transfection of PBAE/mRNA KNPs among homing monocytes;

FIG. 9 demonstrates the addition of 2.5 mM sodium citrate successfully induced particle size growth; and

FIG. 10A, FIG. 10B, FIG. 10C, and FIG. 10D demonstrate the application of the presently disclosed nanoparticle system for gene editing. FIG. 10A shows that 200-nm particles resulted in around 35% cells transfected, however, only 1.3% cells were edited. FIG. 10B showed that 400-nm particles resulted in about 70% cells transfected (top-right and bottom-right quadrants), which is significantly higher than that of 200-nm particles. Out of these 70% cells, nearly one third were successfully edited. FIG. 10C summarizes the particle size effect and shows that 900-nm particles resulted in similar outcomes as those observed for 400-nm particles. FIG. 10D summarizes the relative efficiency of successful editing to successful transfection.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides hybrid nanoparticles having a defined size in a range between about 50 nm to about 1000 nm prepared by a kinetic assembly process. The presently disclosed hybrid nanoparticles comprise a biodegradable polycation and a PEGylated lipid and include a nucleic acid including, but not limited to, plasmid DNA, messenger RNA (mRNA), small interfering RNA (siRNA), and the like. The assembled hybrid nanoparticles can be used for gene delivery therapy in vivo through various delivery routes and can be used ex vivo and in vitro to transfect cells of interest. The presently disclosed hybrid nanoparticles with certain sizes within a sub-micron range exhibited significantly improved transfection efficiency compared to nanoparticles without size control. Accordingly, the presently disclosed hybrid nanoparticles may accelerate clinical translation of non-viral gene therapies.

### A. Method for Preparing Hybrid Nanoparticles

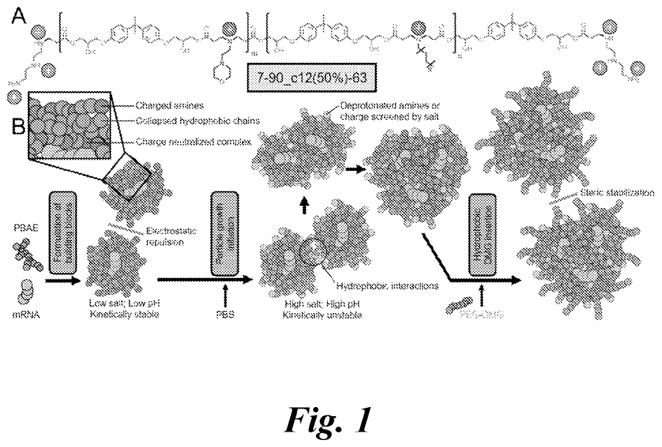

In some embodiments, the presently disclosed method for preparing a hybrid nanoparticle comprising a polycation, a nucleic acid, and a PEGylated lipid. Referring now to FIG. 1, the presently disclosed method generally includes (a) formulation of the building blocks; (b) initiating particle growth; and (c) insertion of a hydrophobic PEGylated lipid.

More particularly, the presently disclosed method includes assembling a polycation, in some embodiments, a poly(beta-amino ester) (PBAE), with a nucleic acid to form a charge-neutralized complex comprising a nucleic acid and a PBAE having charged amine end groups and one or more hydrophobic side chains. Such complexes are kinetically stable under low salt, low pH conditions. Particle growth is initiated by disposing the complexes in PBS. Particle growth is controlled by high salt, high pH conditions. A hydrophobic PEGylated lipid is then inserted into the complex to form stable particles. In some embodiments, the hybrid nanoparticle is tagged or surface modified with a ligand or other reactive group.

More particularly, in some embodiments, the presently disclosed subject matter provides a method for preparing a plurality of hybrid nanoparticles having a defined size, the method comprising:
(a) mixing a biodegradable cationic polymer and one or more nucleic acids to form a first plurality of charge-neutralized complexes;
(b) disposing the plurality of charge-neutralized complexes in a buffer solution for a period of time to induce particle growth to form a second plurality of charge-neutralized complexes, wherein the second plurality of charged neutralized complexes has a particle size greater than a particle size of the first plurality charge-neutralized complexes; and
(c) adding a hydrophobic PEGylated lipid to the second plurality of charge-neutralized complexes to quench particle growth and to form a plurality of hybrid nanoparticles having a defined size.

In some embodiments, the first plurality of charge-neutralized complexes are formed under low salt concentration and low pH conditions. The low salt concentration ranges from an ionic strength equivalent to about 1 mM to about 40 mM NaCl, including about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 mM NaCl, while the high pH ranges from about 2 to about 5.5, including a pH of about 2.0, 2.5, 3.0, 3.5, 4.0, 4.5, 5.0, and 5.5.

In some embodiments, the second plurality of charge-neutralized complexes are formed under high salt concentration and high pH conditions in the presence of one or more multivalent ions. In certain embodiments, the one or more multivalent ions comprise a negatively-charged ion selected from phosphate, citrate, EDTA, pyrophosphate, ATP, tripolyphosphate, and hexametaphosphate. In certain embodiments, the one or more multivalent ions comprise a positively-charged magnesium, calcium, ferrous, and aluminum. In certain embodiments, the high salt concentration ranges from an ionic strength equivalent to about 40 mM to about 300 mM NaCl, including about 40, 50, 60, 70, 80, 90, 100, 110, 120, 130, 140, 150, 160, 170, 180, 190, 200, 210, 220, 230, 240, 250, 260, 270, 280, 290, and 300 mM NaCl, while the high pH ranges from about 5.5 to about 9.0, including a PH of about 5.5, 6.0, 6.5, 7.0, 7.5, 8.0, 8.5, and 9.0.

In some embodiments, the biodegradable cationic polymer and the one or more nucleic acids are mixed through pipetting, at a T junction flow path, in a microfluidic channel or mixer, in a multi-inlet vortex mixer, or in a confined impinging jet (CIJ) mixer. In particular embodiments, the biodegradable cationic polymer and the one or more nucleic acids are mixed in a confined impinging jet (CIJ) mixer.

CIJ mixers and their use are described in International PCT Patent Application No. WO2020223323 for Compositionally Defined Plasmid DNA/Polycation Nanoparticles and Methods for Making the Same, to Mao et al., published Nov. 5, 2020; International PCT Patent Application Publication No. WO2021252715 for Axisymmetric Confined Impinging Jet Mixer, to Mittal et al., published Dec. 16, 2021; and International PCT Patent Application No. PCT/US22/16583 for Methods for Preparation of Shelf-Stable Plasmid DNA/Polycation Particles with Defined Sizes for Cell Transfection, to Mao et al., filed Feb. 16, 2022; each of which is incorporated herein by reference in its entirety

In some embodiments, the plurality of hybrid nanoparticles are prepared by “flash nanocomplexation (FNC).” See, e.g., International PCT Patent Application No. WO2020223323 for Compositionally Defined Plasmid DNA/Polycation Nanoparticles and Methods for Making the Same, to Mao et al., published Nov. 5, 2020, which is incorporated herein by reference in its entirety. In such FNC methods, uniform polyelectrolyte complex (PEC) nanoparticles are continuously generated by: (a) flowing a first stream comprising one or more water-soluble polycationic polymers at a first variable flow rate into a confined chamber; (b) flowing a second stream comprising one or more water-soluble polyanionic polymers at a second variable flow rate into the confined chamber, wherein the first stream and the second stream are on opposing sides when entering the confined chamber; and (c) optionally flowing a third stream comprising one or more components selected from the group consisting of one or more water-soluble therapeutic agents, one or more miscible organic solvents, and/or one or more cryoprotectants at a third variable flow rate into the confined chamber; wherein each stream is equidistant from the other two streams when entering the confined chamber; wherein the first variable flow rate, the second variable flow rate, and the third variable flow rate, if present, can be the same or different; and (d) impinging the first stream, the second stream, and the third stream, if present, in the confined chamber until the Reynolds number is from about 1,000 to about 20,000, thereby causing the one or more water-soluble polycationic polymers and the one or more water-soluble polyanionic polymers to undergo a polyelectrolyte complexation process that continuously generates PEC nanoparticles, wherein the polyelectrolyte complexation process occurs under conditions having a characteristic assembly time (TA), over which assembly of the PEC nanoparticles occurs, which is greater than a characteristic mixing time (TM), over which components of the first stream, second stream, and third stream, if present, are mixed homogenously.

In representative embodiments, the first variable flow rate, the second variable flow rate, and the third variable flow rate, if present, are each equal to or greater than about 3 milliliters/minute (mL/min). In more particular representative embodiments, the first variable flow rate, the second variable flow rate, and the third variable flow rate, if present, are each between about 3 mL/min to about 50 mL/min. In certain representative embodiments, the characteristic mixing time is between about 1 ms to about 200 ms. In particular embodiments, the characteristic mixing time is about 15 ms. In some embodiments, the Reynolds number has a range from about 2,000 to about 8,000 or from about 3,000 to about 5,000. In some embodiments the pH value of the first stream and the pH value of the second stream each has a range from about 2.5 to about 8.4. In particular embodiments, the pH value of the first stream and the pH value of the second stream each is about 3.5.

In certain embodiments, the biodegradable cationic polymer and the one or more nucleic acids are mixed at a pH of about 5.

In certain embodiments, the biodegradable cationic polymer and the one or more nucleic acids are mixed at a ratio of about 3:1 nucleic acid:cationic polymer.

In certain embodiments, the buffered solution comprises phosphate buffered saline (PBS). In particular embodiments, the phosphate buffered saline comprises one or more of NaCl, KCl, Na₂HPO₄, KH₂PO₄, and combinations thereof.

In certain embodiments, the period of time the first plurality of charge-neutralized complexes is disposed in the buffer solution has a range from about 0.1 min to about 300 min. In particular embodiments, the period of time the first plurality of charge-neutralized complexes is disposed in the buffer solution has a range selected from about 0.1 min to about 30 min; from about 0.1 min to about 20 min; about 0.1 min to about 19 min; about 0.1 min to about 18 min; about 0.1 min to about 17 min; about 0.1 min to about 16 min; about 0.1 min to about 15 min; about 0.1 min to about 14 min; about 0.1 min to about 13 min; about 0.1 min to about 12 min; about 0.1 min to about 11 min; about 0.1 min to about 10 min; about 0.1 min to about 9 min; about 0.1 min to about 8 min; about 0.1 min to about 7 min; about 0.1 min to about 6 min; about 0.1 min to about 5 min; about 0.1 min to about 4 min; about 0.1 min to about 3 min; about 0.1 min to about 2 min; and about 0.1 min to about 1 min.

In some embodiments, the PEGylated lipid comprises 1,2-dimyristoyl-sn-glycero-3-methoxypolyethylene glycol (DMG-PEG). In particular embodiments, the PEGylated lipid is DMG-PEG2000. In more particular embodiments, the DMG-PEG2000 comprises greater than about 5% of a mass concentration of the hybrid nanoparticle. In yet more particular embodiments, the DMG-PEG2000 comprises between about 5% to about 20% of the mass concentration of the hybrid nanoparticle, including about 5, 6, 7, 8, 9, 10, 11, 12, 13, 14, 15, 16, 17, 18, 19, and 20% of the mass concentration of the hybrid nanoparticle. In some embodiments, the PEGylated lipid comprises C18-PEG2000.

In some embodiments, the PEGylated lipid further comprises a chemically-active moiety. In certain embodiments, the method further comprises functionalizing the chemically-active moiety with a targeting ligand or other biologically active chemical structures.

In some embodiments, the one or more nucleic acids is selected from an antisense oligonucleotide, cDNA, genomic DNA, guide RNA, plasmid DNA (pDNA), including a mixture of different species of pDNA, vector DNA, mRNA, miRNA, piRNA, shRNA, and siRNA. In particular embodiments, the one or more nucleic acids comprises mRNA. In more particular embodiments, the plurality of hybrid nanoparticles comprises between about 2 to about 1500 copies of mRNA per particle, including about 2, 3, 4, 5, 6, 7, 8, 9, 10, 50, 100, 200, 300, 400, 500, 600, 700, 800, 900, 1000, 1100, 1200, 1300, 1400, and 1500, and any integer in between.

In some embodiments, the first plurality of charge-neutralized complexes has a particle size having a range between about 50 nm to about 120 nm, including about 50, 55, 60, 65, 70, 75, 80, 85, 90, 95, 100, 105, 110, 115, and 120 nm.

In some embodiments, the plurality of hybrid nanoparticles have an average particle size having a range from about 50 nm to about 1000 nm; about 50 nm to about 900 nm; about 50 nm to about 800 nm; about 50 nm to about 700 nm; about 50 nm to about 600 nm; about 50 nm to about 500 nm; about 50 nm to about 400 nm; about 50 nm to about 300 nm; about 50 nm to about 200 nm; and about 50 nm to about 100 nm. In particular embodiments, the plurality of hybrid nanoparticles have an average particle size of about 400 nm, including about 350 nm, 355 nm, 360 nm, 365 nm, 370 nm, 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, and 450 nm.

In some embodiments, the plurality of hybrid nanoparticles have a zeta-potential of between about 2 mV and about 6 mV, including about 2 mV, 2.5 mV, 3 mV, 3.5 mV, 4 mV, 4.5 mV, 5 mV, 5.5 mV, and 6 mV.

In some embodiments, the plurality of hybrid nanoparticles have an encapsulation efficiency of between about 80% to about 100%, including about 80, 81, 82, 83, 84, 85, 86, 87, 88, 89, 90, 91, 92, 93, 94, 95, 96, 97, 98, 99, 99.5, 99.9, and 100% encapsulation efficiency.

In some embodiments, the presently disclosed subject matter provides a biodegradable cationic polyester for delivery of nucleic acids. As used herein, “biodegradable” polymers and/or nanoparticles are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). Such components preferably do not induce inflammation or other adverse effects in vivo. In some instances, the chemical reactions relied upon to break down the biodegradable compounds are uncatalyzed.

In certain embodiments, the biodegradable polymers and/or nanoparticles comprise a chemical moiety having one or more degradable linkages, such as an ester linkage, a disulfide linkage, an amide linkage, an anhydride linkage, and a linkage susceptible to enzymatic degradation. Representative degradable linkages include, but are not limited to:

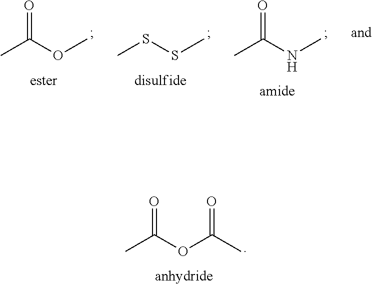

In some embodiments, the biodegradable polymer and/or nanoparticle comprises a poly(beta-amino ester) (PBAE). Exemplary PBAEs suitable for use with the presently disclosed subject matter include those disclosed in:
U.S. Pat. No. 9,884,118 for Multicomponent Degradable Cationic Polymers, to Green et al., issued Feb. 6, 2018;
U.S. Pat. No. 9,802,984 for Biomimetic Peptide and Biodegradable Delivery Platform for the Treatment of Angiogenesis- and Lymphangiogenesis-Dependent Diseases, to Popel et al., issued Oct. 31, 2017;
U.S. Pat. No. 9,717,694 for Peptide/Particle Delivery Systems, to Green et al., issued Aug. 1, 2017;
U.S. Pat. No. 8,992,991 for Multicomponent Degradable Cationic Polymers, to Green et al., issued Mar. 31, 2015;
U.S. Patent Application Publication No. 20180256745 for Biomimetic Artificial Cells: Anisotropic Supported Lipid Bilayers on Biodegradable Micro and Nanoparticles for Spatially Dynamic Surface Biomolecule Presentation, to Meyer et al., published Sep. 13, 2018;
U.S. Patent Application Publication No. 20180112038 for Poly(Beta-Amino Ester)-Co-Polyethylene Glycol (PEG-PBAE-PEG) Polymers for Gene and Drug Delivery, to Green et al., published Apr. 26, 2018;
U.S. Patent Application Publication No. 20170216363 for Nanoparticle Modification of Human Adipose-Derived Mesenchymal Stem Cells for Treating Brain Cancer and other Neurological Diseases, to Quinones-Hinojosa and Green, published Aug. 3, 2017;
U.S. Patent Application Publication No. 20150273071 for Bioreducible Poly(Beta-Amino Ester) s For siRNA Delivery, to Green et al., published Oct. 1, 2015;
U.S. Pat. No. 8,287,849 for Biodegradable Poly(beta-amino esters) and Uses Thereof, to Langer, et al., issued Oct. 16, 2012;
International PCT Patent Application Publication No. WO2020198145 for Gene Delivery Particles to Induce Tumor-Derived Antigen Presenting Cells, to Green, published Oct. 1, 2020; and
International PCT Patent Application Publication No. WO2022067249 for Polymers and Nanoparticle Formulations for Systemic Nucleic Acid Delivery, to Green et al., published Mar. 31, 2022; each of which is incorporated by reference in their entirety.

Generally, the presently disclosed multicomponent degradable cationic polymers include a backbone derived from a diacrylate monomer (designated herein below as “B”), an amino-alcohol hydrophilic side-chain monomer or a hydrophobic side-chain monomer (designated herein below as “S”), and an amine-containing endcapping monomer (designated herein below as “E”). The endcapping group structures are distinct and separate from the polymer backbone structures and the side chain structures of the intermediate precursor molecule for a given polymeric material. In particular embodiments, the side-chain monomer comprises a hydrophobic side-chain monomer.

The presently disclosed PBAE compositions can be designated, for example, as B5-S4-E7 or 547, in which R is B5, R′ is S4, and R″ is E7, and the like, where B is the backbone and S is the side chain, followed by the number of carbons in their hydrocarbon chain, e.g., S4 comprises 4 alkylene groups. Endcapping monomers, E, are sequentially numbered according to similarities in their amine structures. Further, in particular embodiments, the presently disclosed PBAE includes a hydrophobic side-chain, which is designated SC-XX, with XX being the number of carbon atoms in the chain.

To form the compounds of formula (I), acrylate monomers can be condensed with amine-containing side chain monomers. In some embodiments, the side-chain monomers comprise a primary amine, but, in other embodiments, the side-chain monomers comprise a secondary or a tertiary amine. Side chain monomers may further comprise a C₁ to C₈ linear or branched alkylene, which is optionally substituted. Illustrative substituents include hydroxyl, alkyl, alkenyl, thiol, amine, carbonyl, and halogen. Acrylate terminated polymers can be synthesized from small molecule diacrylate and primary amine monomers followed by endcapping with R″ monomers. In certain embodiments, the linear and/or branched PBAE polymer has a molecular weight of from 5 to 10 kDa, or a molecular weight of from to 15 kDa, or a molecular weight of from 15 to 25 kDa, or a molecular weight of from 25 to 50 kDa.

In some embodiments, the biodegradable cationic polymer comprises a poly(beta-amino ester) (PBAE). In certain embodiments, the PBAE comprises a compound of formula (I):

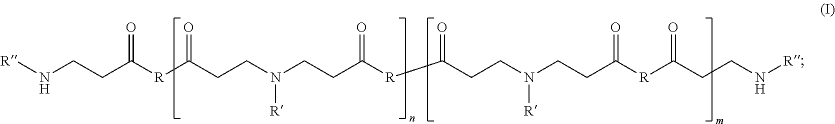

wherein:
m and n are each independently an integer from 1 to 10,000;
R comprises a divalent radical comprising a biodegradable ester linkage and/or a bioreducible disulfide linkage;
each R′ can be the same or different and is selected from a hydrophobic sidechain or a hydrophilic sidechain comprising a monovalent radical derived from an amine monomer;
R″ is monovalent radical derived from an amine-containing end capping group; and pharmaceutically acceptable salts thereof.

In certain embodiments, R is selected from:

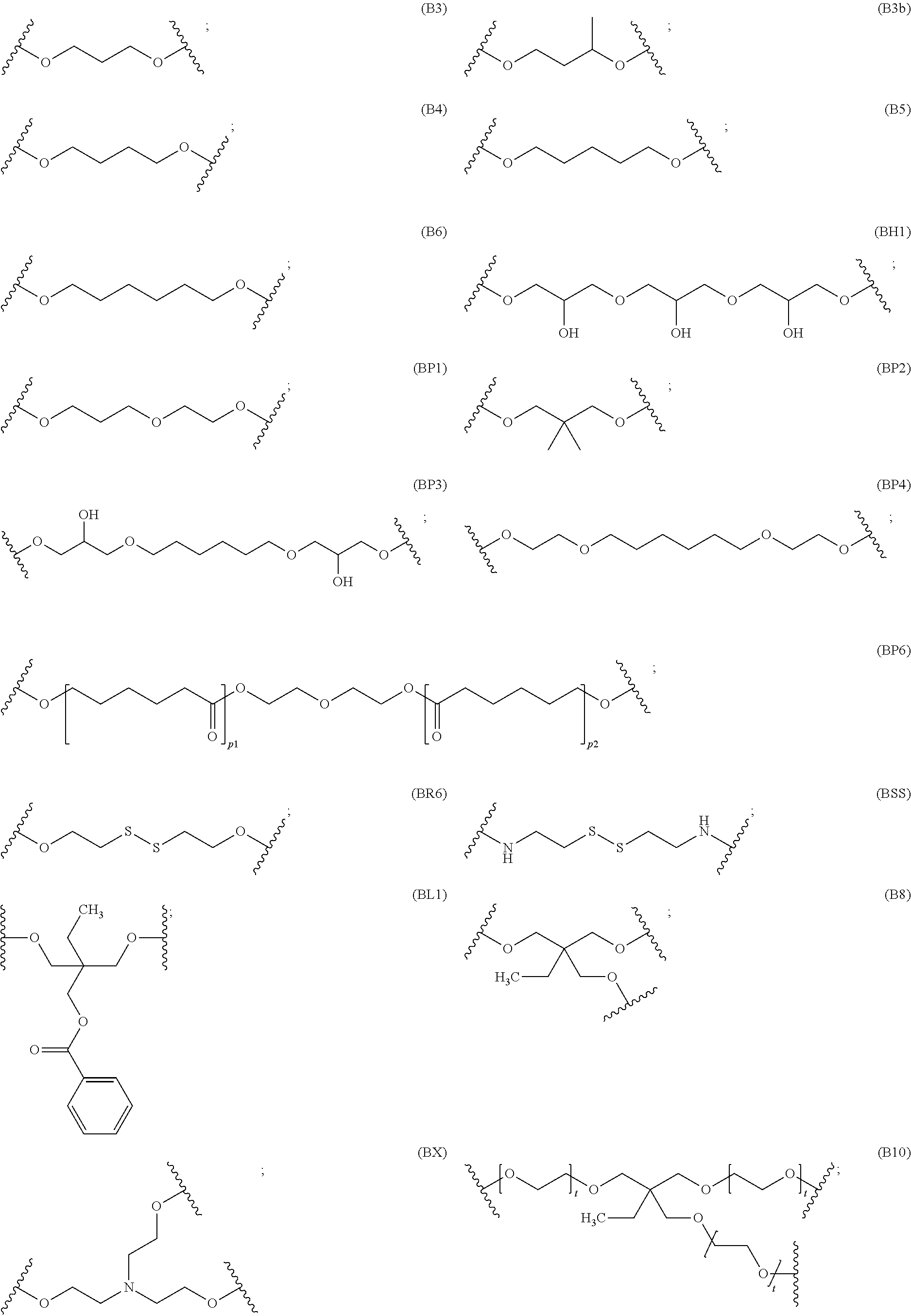

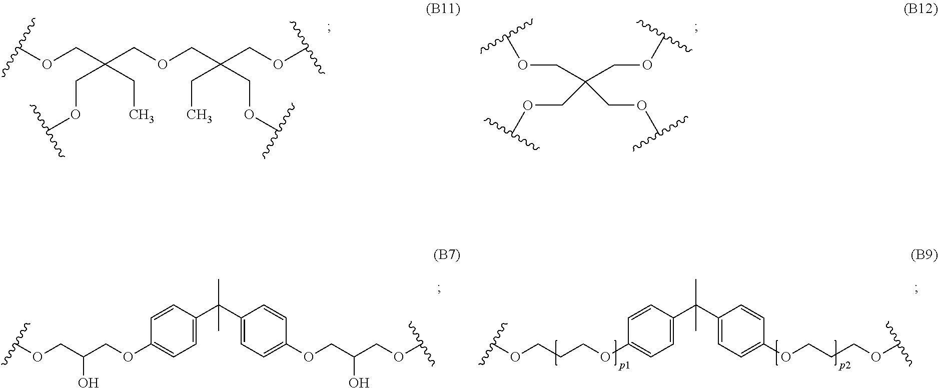

wherein each p1, p2, and t is independently an integer selected from the group consisting of 1, 2, 3, 4, 5, 6, 7, 8, 9, and 10.

In particular embodiments, each R is:

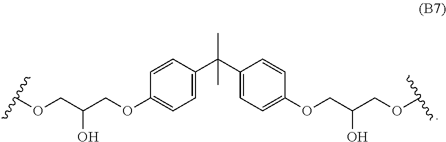 [SMILES: *OCC(O)COc1ccc(C(C)(C)c2ccc(OCC(O)CO*)cc2)cc1]

In some embodiments, at least one R′ comprises:

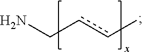

wherein x is an integer selected from 0, 1, 2, 3, 4, 5, 6, 7, 8, 9, 10, 11, 12, 13, 14, 15, 16, 17, 18, 19, and 20; and wherein

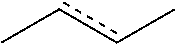

can be a single or double bond in one or more x repeating units. In certain embodiments, at least one R′ is selected from:

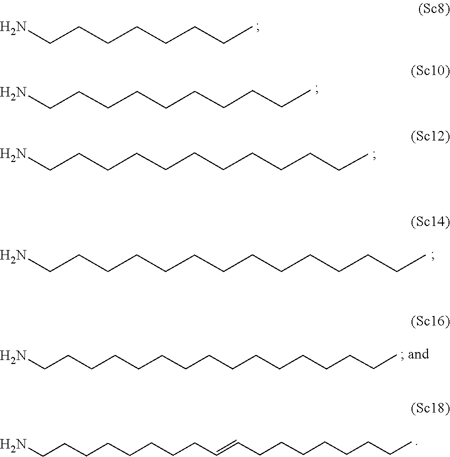

In certain embodiments, at least one R′ is a hydrophilic sidechain selected from:

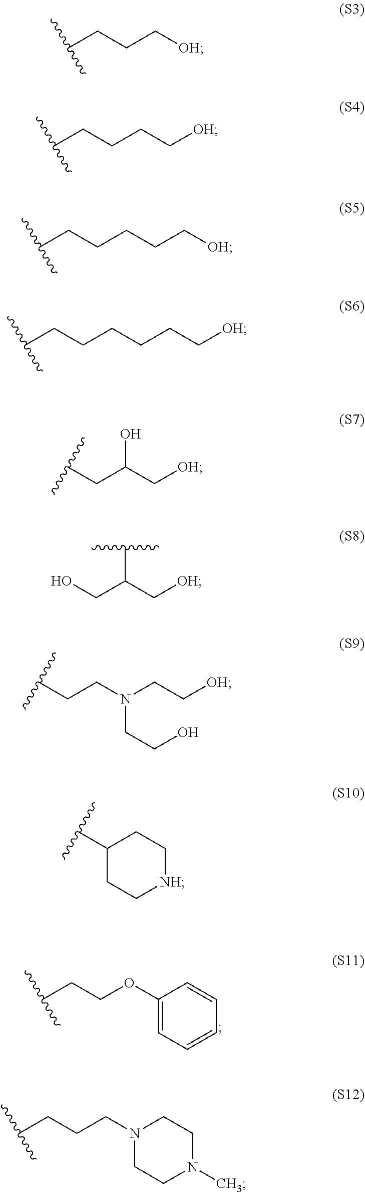

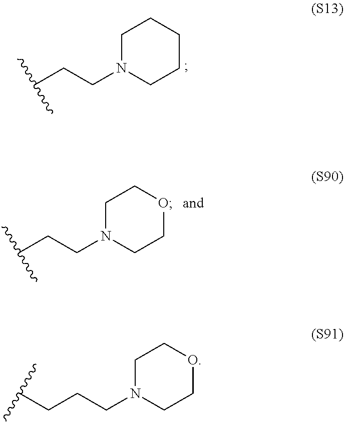 [SMILES: *CCN1CCCCC1 ; *CCN1CCOCC1 ; "and" ; *CCCN1CCOCC1]

In particular embodiments, at least one R′ is:

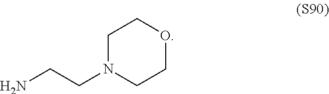

In certain embodiments, R″ is selected from the group consisting of:

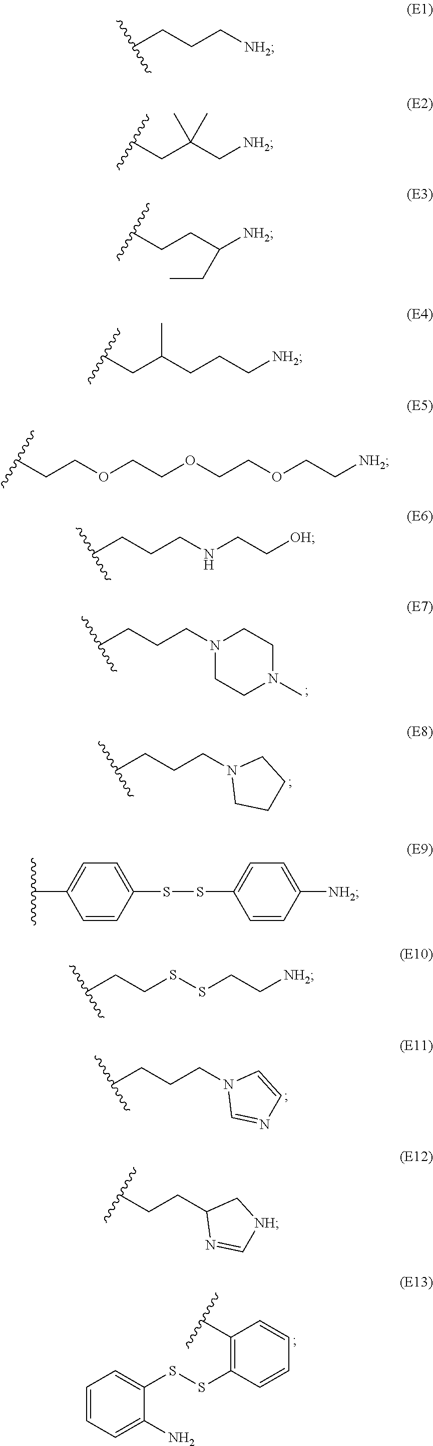

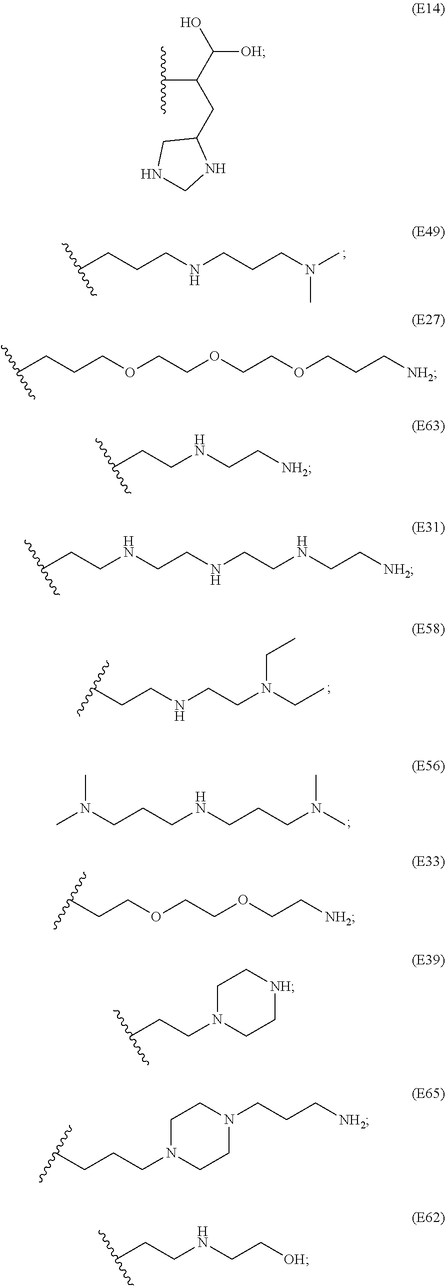

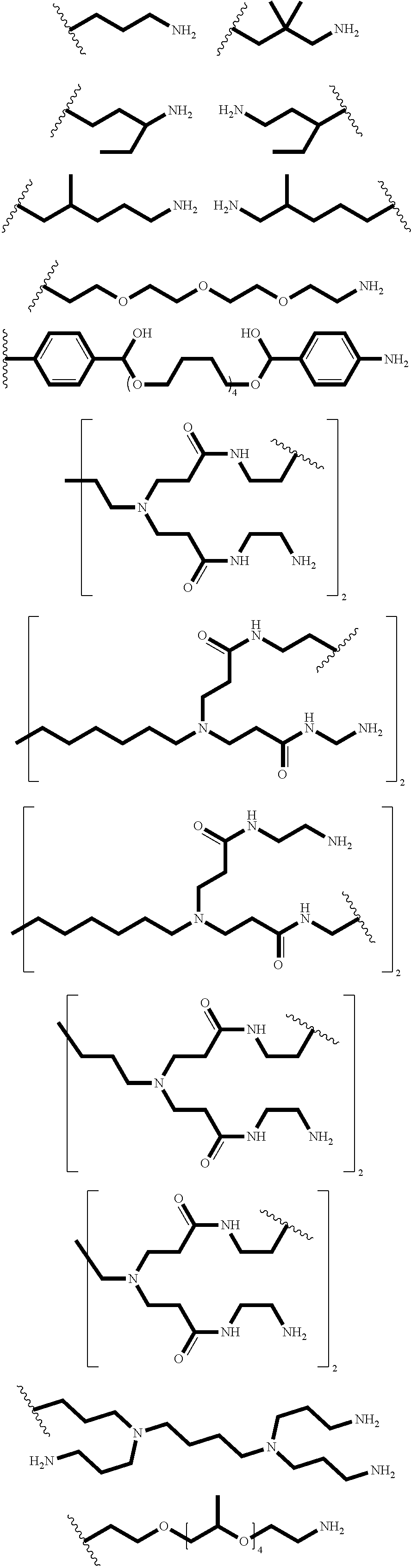

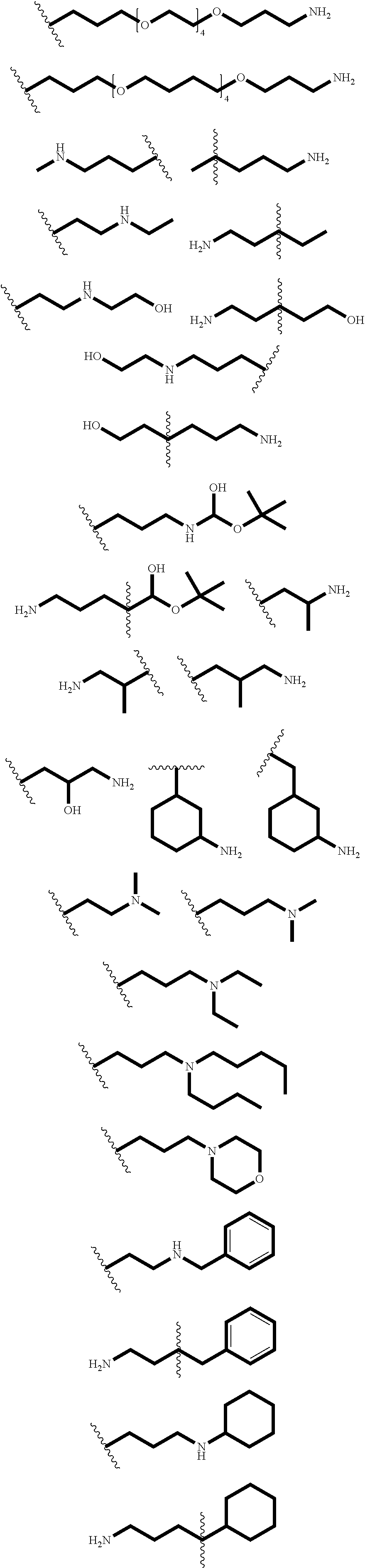

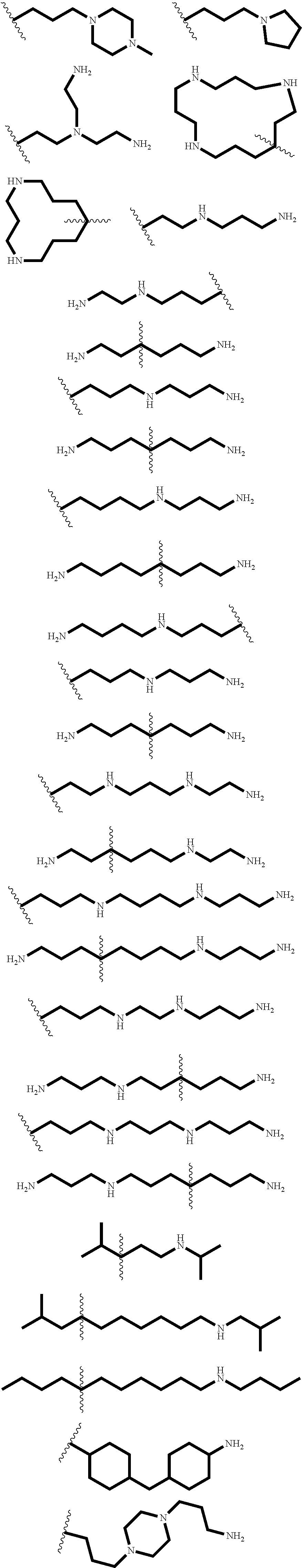

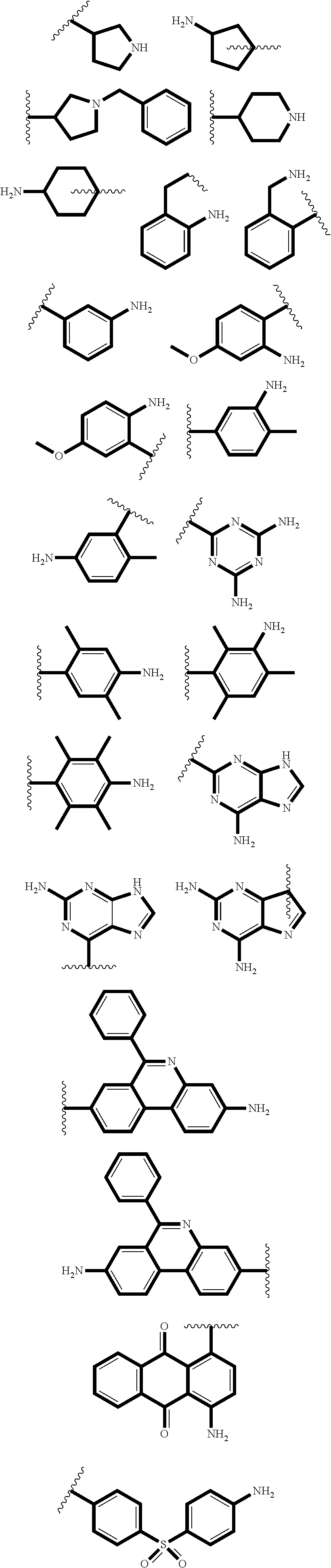

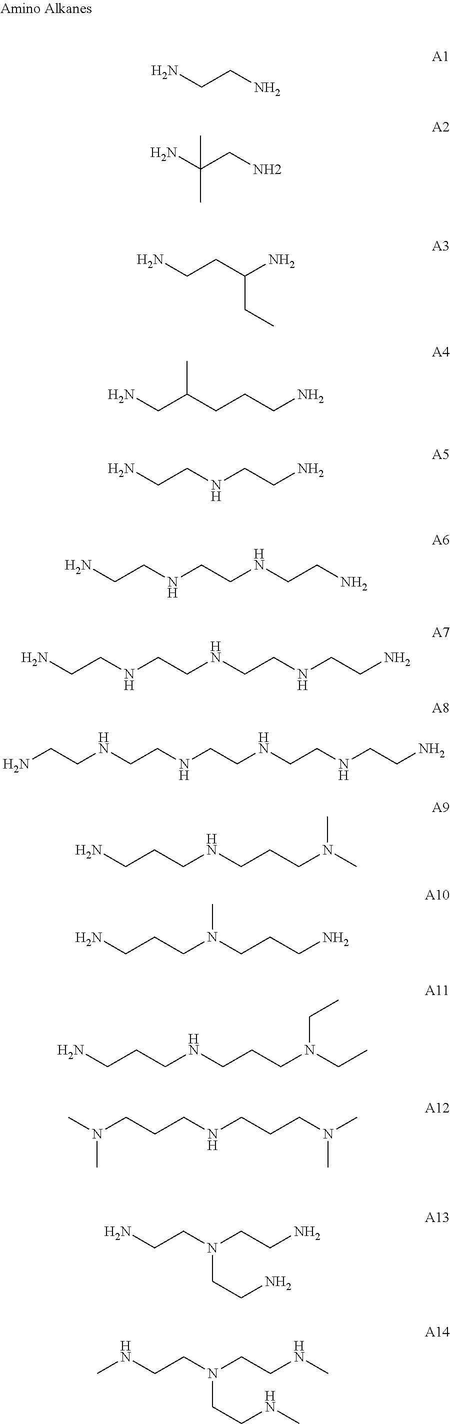

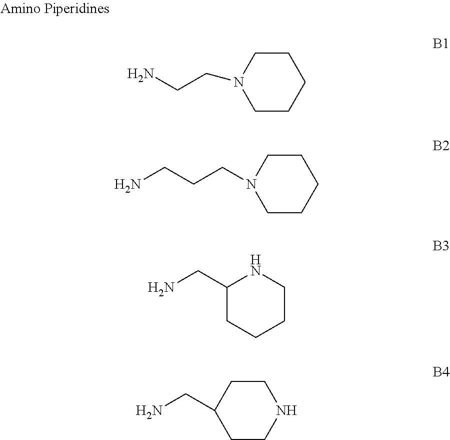

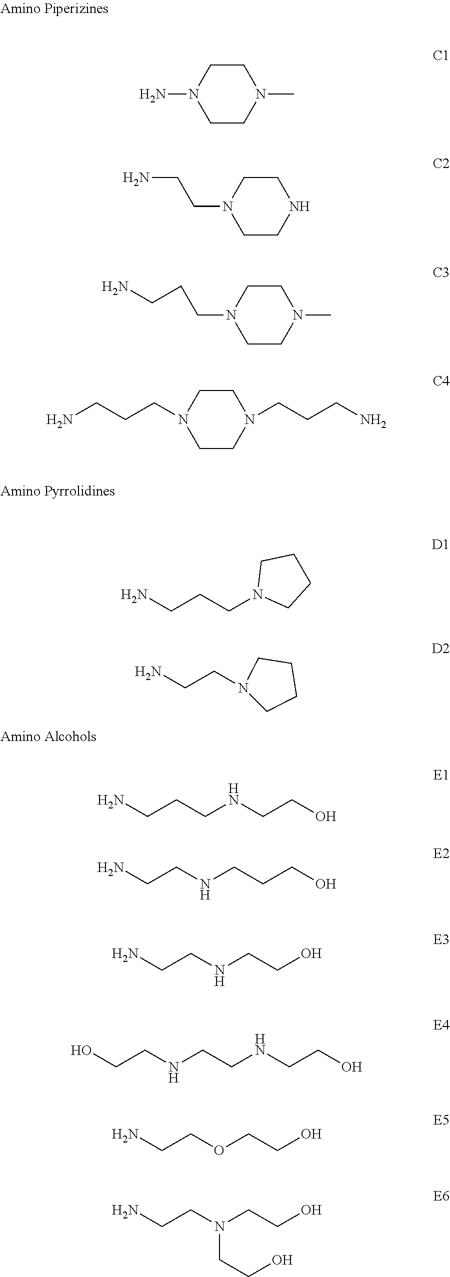

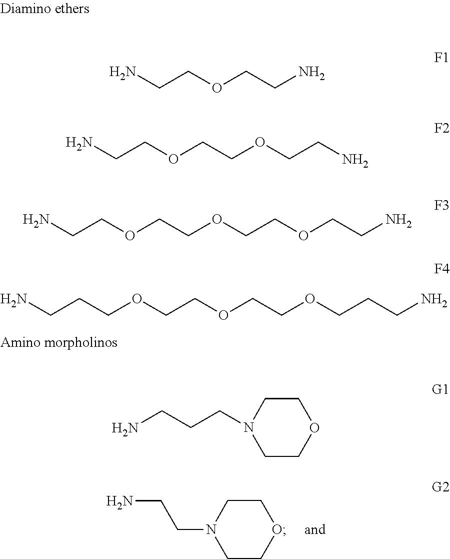

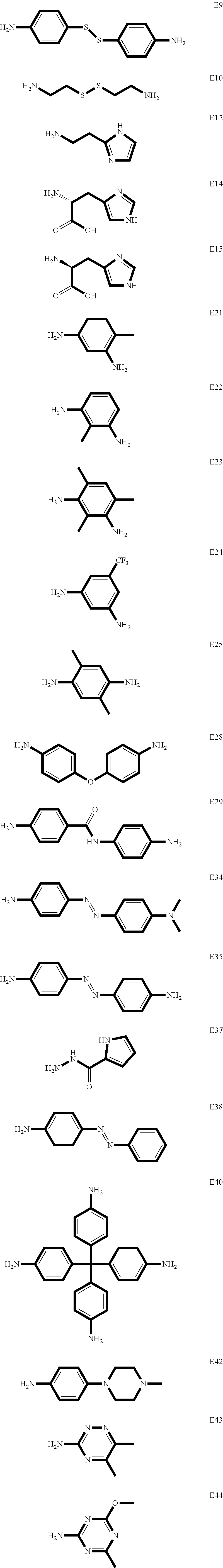

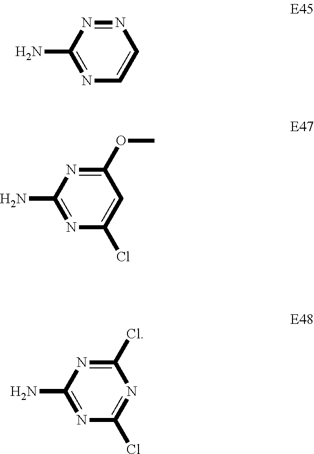

In particular embodiments, R″ is:

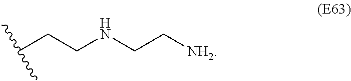 [SMILES: *CCNCCN]

In some embodiments, the linear diacrylate R is B7, the end-capping group R″ is E63, one R′ is a hydrophilic amine comprising S90, and the other R′ is a hydrophobic amine selected from the group consisting of S8, S10, S12, S14, S16, and S18. In some embodiments, at least one of S8, S10, S12, S14, S16, and S18 is present at a percentage ranging from about 15% to 80% relative to a percentage of S90, including about 15, 20, 25, 30, 35, 40, 45, 50, 55, 60, 65, 70, 75, and 80% relative to a percentage of S90.

In yet more particular embodiments, the compound of formula (I) is:

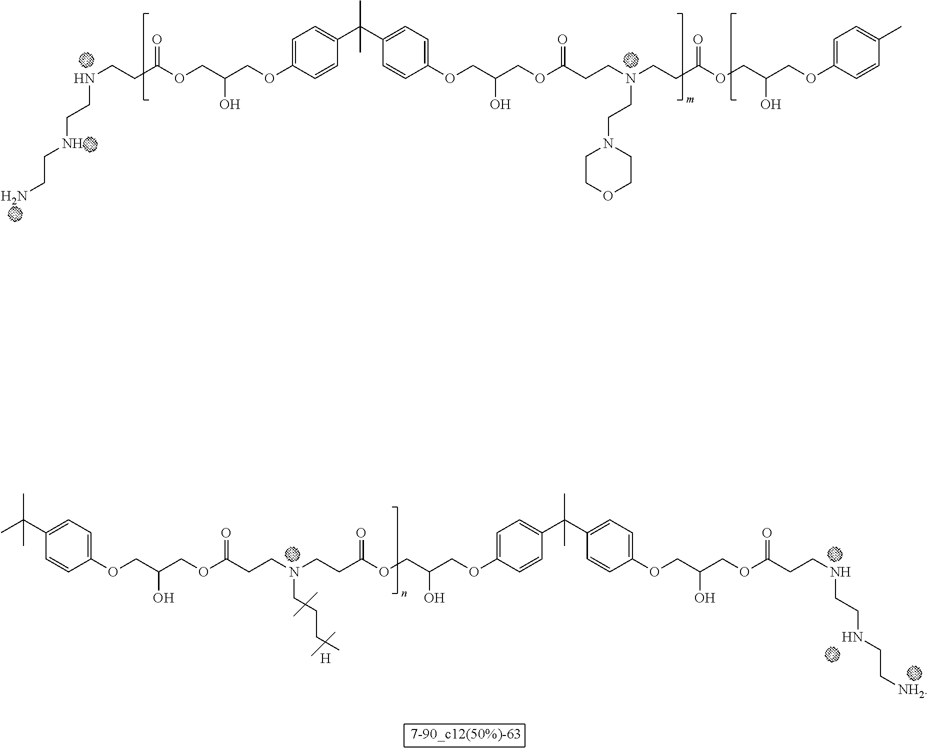

In some embodiments, the method further comprises an excipient for cryo-preservation of the plurality of particles. In certain embodiments, the excipient is selected from a saccharide and a sugar alcohol. In particular embodiments, the saccharide is selected from a monosaccharide, a disaccharide, and a polysaccharide. In particular embodiments, the sugar alcohol is selected from arabinose, glucose, fructose, ribose, mannose, sucrose, trehalose, lactose, maltose, starches, dextran, mannitol, and sorbitol. In more particular embodiments, the sugar alcohol is 15% w/v trehalose. In some embodiments, the method further comprises lyophilizing or freezing the particles at about −80° C. for storage.

### B. Hybrid Nanoparticles

In some embodiments, the presently disclosed subject matter provides a hybrid nanoparticle comprising a PBAE, a nucleic acid, and a PEGylated lipid, wherein nucleic acid comprises mRNA, the PEGylated lipid comprises DMG-PEG2000, and the PBAE comprises a compound of the following formula:

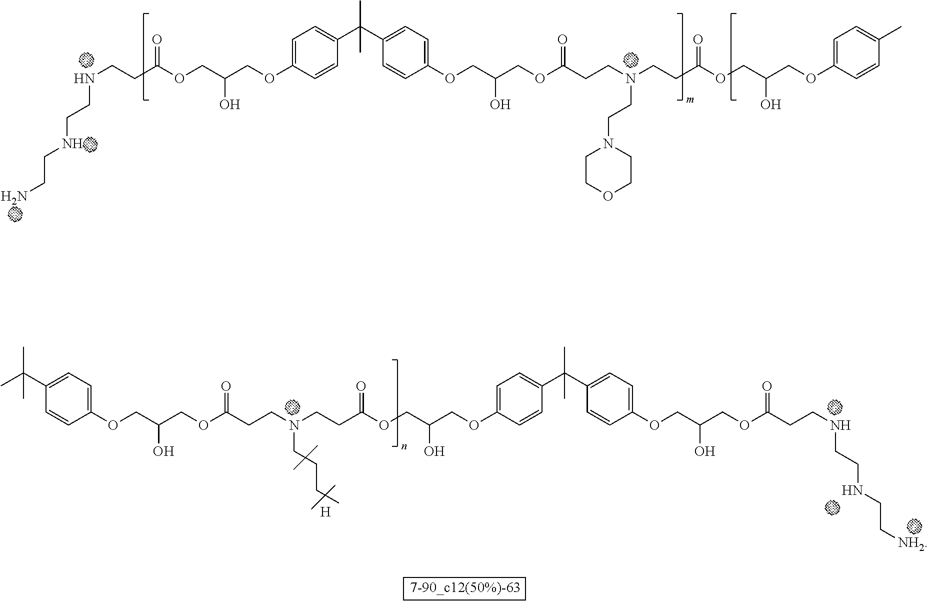

In certain embodiments, the hybrid nanoparticle has particle size of about 400 nm. In certain embodiments, the DMG-PEG2000 comprises between about 5% to about 20% of a mass concentration of the nanoparticle. In certain embodiments, the hybrid nanoparticle comprises between about 2 to about 1500 copies of mRNA per particle. In certain embodiments, the plurality of hybrid nanoparticles have a zeta-potential of between about 2 mV and about 6 mV. In certain embodiments, the hybrid nanoparticle has an encapsulation efficiency of between about 80% to about 100%. In certain embodiments, the PEGylated lipid further comprises a chemically-active moiety. In certain embodiments, the chemically-active moiety is functionalized with a targeting ligand or other biologically active chemical structure.

In some embodiments, the presently disclosed subject matter provides a formulation comprising the presently disclosed composition, wherein the formulation is one or more of frozen, lyophilized, or combined with one or more excipients to extend stability.

For example, in some embodiments, the presently disclosed subject matter also includes a method of using and storing the polymers and particles described herein whereby a cryoprotectant (including, but not limited to, a sugar) is added to the polymer and/or particle solution and it is lyophilized and stored as a powder. Such a powder is designed to remain stable and be reconstituted easily with aqueous buffer as one skilled in the art could utilize. Moreover, freeze-dried nanoparticles typically are stable for up to two years when stored at room temperature, 4° C., or −20° C. In some embodiments, the composition is lyophilized, and reconstituted prior to administration to a subject, e.g. a patient.

Depending on the specific conditions being treated, the pharmaceutical composition may be formulated into liquid or solid dosage forms and administered systemically or locally. The pharmaceutical composition may be delivered, for example, in a timed- or sustained-low release form as is known to those skilled in the art. Techniques for formulation and administration may be found in “Remington: The Science and Practice of Pharmacy (20th ed.)” Lippincott, Williams & Wilkins (2000). Suitable routes may include oral, buccal, by inhalation spray, sublingual, ocular, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, intratumoral, intraocular (e.g., intravitreal) injections, or other modes of delivery.

While the form and/or route of administration can vary, in some embodiments the pharmaceutical composition is formulated for parenteral administration (e.g., by subcutaneous, intravenous, or intramuscular administration).

Formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol.

Use of pharmaceutically acceptable inert carriers to formulate pharmaceutical compositions disclosed herein into dosages suitable for systemic administration is within the scope of the present invention. With proper choice of carrier and suitable manufacturing practice, the compositions of the present invention, in particular, those formulated as solutions, may be administered parenterally, such as by intravenous injection, or locally, such as intraocular injection. The pharmaceutical compositions can be formulated readily using pharmaceutically acceptable carriers well known in the art into dosages suitable for oral administration. Such carriers enable the pharmaceutical composition to be formulated as tablets, pills, capsules, liquids, gels, syrups, slurries, suspensions and the like, for oral ingestion by a subject (e.g., patient) to be treated.

For injection, pharmaceutical compositions of the present invention may be formulated and diluted in aqueous solutions, such as in physiologically compatible buffers such as Hanks' solution, Ringer's solution, or physiological saline buffer.

In certain embodiments, the presently disclosed subject matter provides a pharmaceutical formulation of comprising the presently disclosed compositions in a pharmaceutically acceptable carrier. As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art.

C. Methods for Transfecting Cells, Delivering mRNA to Tissues, Gene Delivery, and Treating a Disease, Condition, or Disorder

In some embodiments, the presently disclosed subject matter provides a method for transfecting a cell, the method comprising administering to the cell a presently disclosed hybrid nanoparticle. In certain embodiments, the method comprises ex vivo or in vitro transfection and gene editing.

In some embodiments, the presently disclosed subject matter provides a method for delivering mRNA to a tissue, the method comprising administering to the tissue a presently disclosed hybrid nanoparticle. In certain embodiments, the tissue is selected from lung, liver, kidney, heart, and spleen.

In some embodiments, the presently disclosed subject matter provides a method for delivering a gene to a subject, the method comprising administering a hybrid nanoparticle to the subject. In certain embodiments, the administration is in vivo. In particular embodiments, the method comprises intravenous administration.

In some embodiments, the presently disclosed subject matter provides a method for treating a disease, condition, or disorder, the method comprising administering to a subject in need of treatment thereof, a presently disclosed hybrid nanoparticle. In certain embodiments, the disease, condition, or disorder comprises cancer.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Supra-Molecular Assembly Using mRNA and Poly(Beta-Amino Ester) (PBAE)

As provided in FIG. 1A and FIG. 1B, particle stabilization relies on insertion of a lipid component (in some embodiments, DMG-PEG) onto the surface of the particles, instead of rendering a low pH to the solution. The insertion of the lipid component ensures particle stability under physiological pH, and a neutral particle surface charge, which are essential for in vivo applications.

In some embodiments, the presently disclosed size control method includes one or more steps of the following representative protocol:
(a) mRNA is dissolved in a MgAc₂ buffer at a pH of 5;
(b) PBAE is dissolved in a blend solution of 90% EtOH and 10% MgAc₂ buffer at a pH of 5;
(c) The mRNA solution and the PBAE solution are mixed at a ratio of 3:1 to form small nanoparticles, through pipetting, a T junction flow path, in a microfluidic channel or mixer, in a multi-inlet vortex mixer, or a confined impinging jet (CIJ mixer). The mRNA concentration is 100 μg/mL and the PBAE concentration is 3 mg/ml;
(e) The nanoparticles were dialyzed against 2.78 mM MgAc₂ buffer with a 1:50 volume ratio at pH=5 to remove EtOH and dilute acidic buffer concentration;
(f) The nanoparticles are mixed with an equal volume of 2× PBS containing 10 mM sodium citrate (or 1× PBS containing 10 mM sodium citrate (plus 15% w/v trehalose when cryo-preservation of the particles is required) to induce the particle size growth;
(g) When the desired particle size is reached, a PEGylated lipid EtOH solution (DMG-PEG2000) is added into the particle mixture to quench the particle growth;
(h) The particles are then stabilized. The quantity of the DMG-PEG2000 is typically 10% of the PBAE mass concentration; and
(i) If the PEGylated lipid carries a chemically active moiety, the size-controlled particles can further be functionalized by a targeting ligands or other biologically active chemical structures.

### Example 2

### In Situ Generation of CAR Cells for Cancer Therapy

Chimeric antigen receptor (CAR) cells have revolutionized the treatment of serious hematologic malignancies in recent years. Waldman et al., 2020. There are currently six US FDA approved CAR therapies, all utilizing autologous T cells: KYMRIAH®, YESCARTA®, TECARTUS®, and BREYANZI® target B cell lymphoma, and ABECMA® and CARVYKTI™ target multiple myeloma. National Cancer Institute 2022a. These engineered cell therapies enable long term remissions and even cure of heretofore refractory cancers. Despite the revolution they have heralded, CAR-T therapies as currently practiced are hampered by serious limitations-slow and costly ex vivo expansion, poor efficacy against solid tumors, and safety concerns. Patel et al., 2019.

The presently disclosed subject matter, in part, leverages recent breakthroughs in RNA nanoparticle composition and methods of manufacture to surmount these obstacles and produce in vivo cancer cell therapies. More particularly, using the presently disclosed kinetic nanoparticles (referred to herein as “KNPs”), chimeric antigen receptor-expressing macrophages (CAR-M) can be made in vivo, a powerful approach that can greatly extend the reach and applicability of this promising cell therapy.

Chimeric antigen receptors (CARs) are protein constructs consisting of an extracellular single chain variable fragment domain and an intracellular domain. These domains recognize a specific antigen overly expressed by cancer cells and provide the necessary signaling to elicit cytotoxic function, respectively. Waldman et al., 2020. CAR therapies typically require immune cells isolated from a patient or donor's blood that are genetically modified to express the construct. The CAR-expressing cells are then grown to a sufficient population and subsequently reinfused into a patient, imposing major cost and efficacy barriers. Parayath and Stephan, 2021. For example, the process of ex vivo CAR expansion leads to significant immune cell exhaustion. Tumeh et al., 2010. Further, CAR-T cells specifically have shown limited efficacy against the solid tumors that constitute a majority of cancer burden. National Cancer Institute, 2022b.

Recently, transfection of CAR constructs into other cytotoxic immune cell types, such as CAR natural killer cells (CAR-NKs) and CAR-Ms, has gained considerable attention. Patel et al., 2019. NK cells, however, are difficult to expand ex vivo, do not effectively penetrate solid tumors, and are prone to suppression. The low abundance of T and NK cells in peripheral blood makes them scarce targets for in vivo generation of these therapeutic cells.

CAR-Ms on the other hand offer distinctive advantages including higher ability of infiltration into solid tumors, synergistic response with endogenous T cells by presenting antigens outside of the CAR target, reduced susceptibility to suppression, short circulating half-life limiting chronic side effects, and higher abundance in blood and tissue, making them attractive targets for in vivo programming. Patel et al., 2019. In addition, like CAR-NKs, CAR-Ms also synergize with monoclonal antibody therapy. No approach to date, however, has enabled efficient in vivo generation of CAR-M therapies, overcoming the cost, safety, and efficacy barriers of existing CAR-Ts.

A kinetically assembled biodegradable nanoparticle (in short, kinetic nanoparticles, KNPs) platform has been developed, thereby permitting the generation of poly(β-amino ester) (PBAE)/mRNA particles that selectively and efficiently deliver genetic cargo to macrophages without the use of targeting ligands. This disruptive technology has the potential to revolutionize cancer therapeutics via in vivo generation of CAR-Ms that addresses current challenges with existing CAR cell therapy products. Previous attempts to generate CAR-M cells in vivo with vehicles decorated with ligands targeting mannose receptors, SIRPα, or CD47 have resulted in limited success, largely due to the inefficient transfection of the relevant cell types. Kang et al., 2021 (demonstrating the generation of CAR-Ms via intratumor injection of mannose-decorated vehicle). The approach disclosed by Kang et al., 2021, however, is challenging for clinical translation as solid tumors are not always readily accessible and targeting ligands tend to be masked by the protein corona in blood. In contrast, the presently disclosed KNP platform is the first to target circulating monocytes and macrophages by intravenous injection without surface modification of the vehicle.

The presently disclosed methods optimize the size and chemical composition of these KNP nanoparticles, which allows limitations in this field to be overcome for the first time. J To this end, the presently disclosed PBAE/mRNA KNP platform offers several advantages over the current state of the art, including high efficiency, ligand-free delivery of genetic cargo at a high payload capacity to monocytes/macrophages in vivo, enabling rapid generation of anti-tumor CAR-Ms by transfecting circulating monocytes. This platform combines the advantages of biodegradable PBAE carrier with a versatile profile for various nucleic acid payloads in different organs and tissues, Karlsson et al., 2020, and a highly controlled nanoparticle manufacturing process referred to as “flash nanocomplexation (FNC).” See, e.g., International PCT Patent Application No. WO2020223323 for Compositionally Defined Plasmid DNA/Polycation Nanoparticles and Methods for Making the Same, to Mao et al., published Nov. 5, 2020, which is incorporated herein by reference in its entirety.

Using this platform, PBAE/mRNA KNPs were engineered with exquisite control over size, with a size ranging between about 200 nm and about 1000 nm, with 400-nm KNPs tailored for in vivo generation of CAR-Ms. These KNPs carry more than 100-fold greater mRNA payloads than the standard size nanoparticles (approximately 100 nm) and permit co-delivering multiple cargos for cell programming.

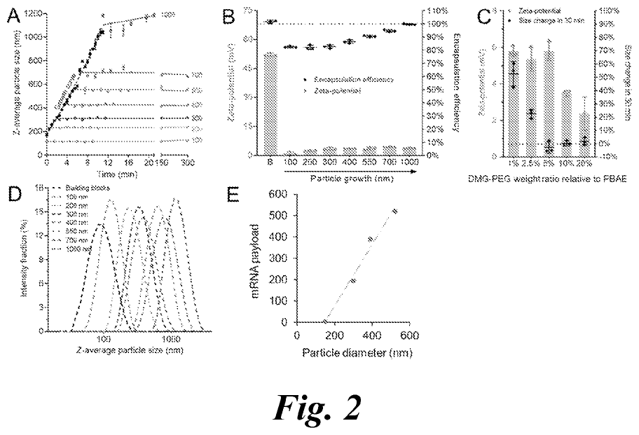

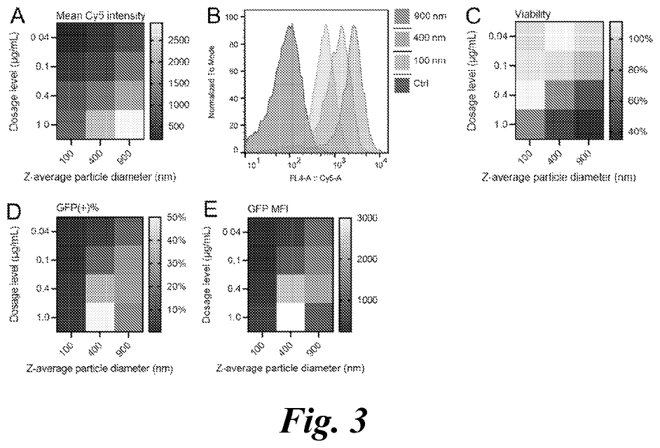

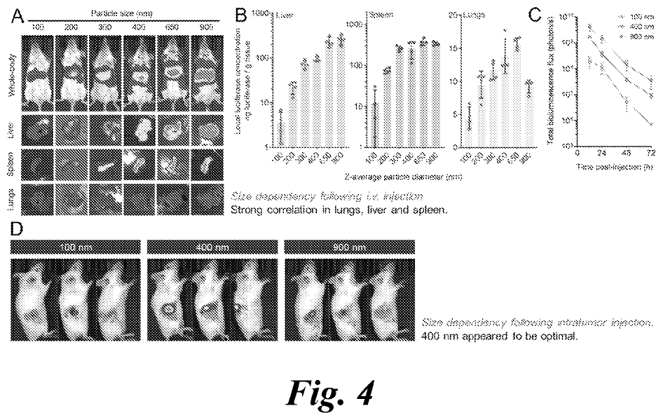

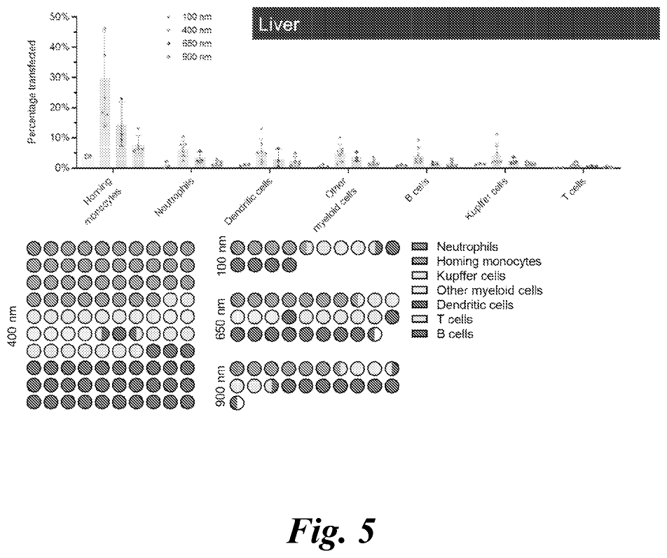

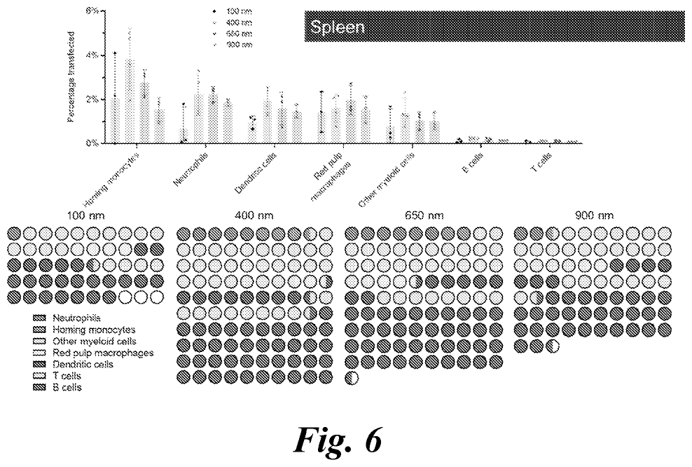

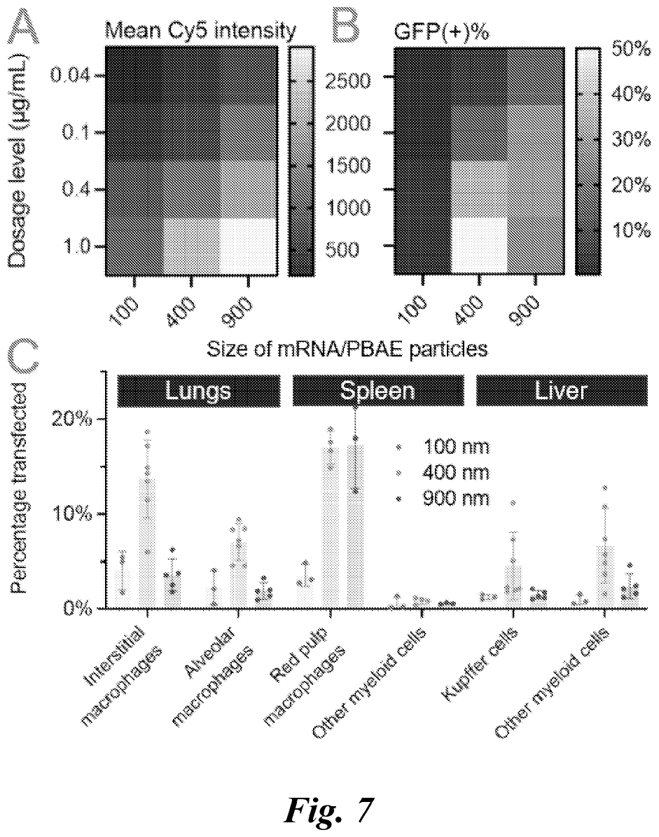

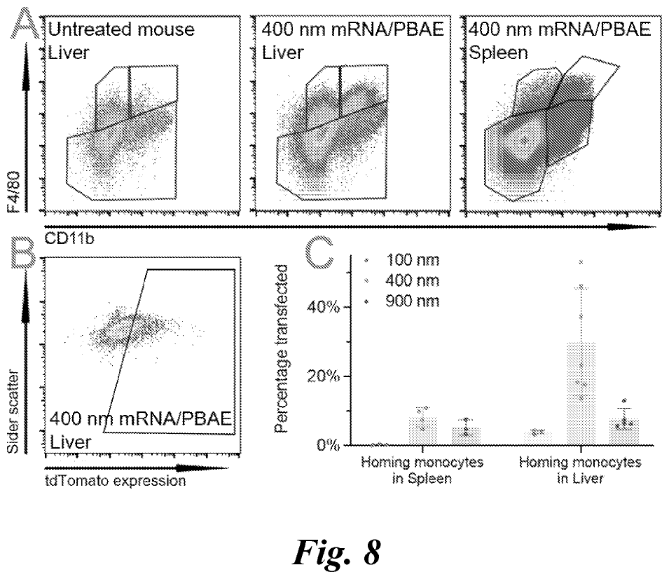

These 400-nm PBAE/mRNA KNPs further exhibit several unique features of that make them optimal for in vivo CAR-M generation. The size optimized KNPs allow high transfection efficiency in bone marrow-derived macrophages (BMDMs), a high transfection efficiency in macrophages in the lung, spleen, and liver following i.v. injection (FIG. 7), more importantly, a high efficiency in transfecting abundant blood-circulating monocytes following i.v. injection, leading to deposition of transfected F4/80⁺/CD11b⁺/CD45⁺/Ly6G⁻ monocytes in the spleen and liver, which were absent without particle treatment (FIG. 2). Therefore, the presently disclosed KNP platform offers a unique opportunity to program the abundant blood-circulating monocytes into CAR-Ms.

Further, these size-controlled particles can be manufactured using a scalable FNC process. The scalability was validated in a 5-L batch production, showing equivalent size control and transfection quality of the particles compared with particles produced at a small bench scale of 50 mL. These results serve as the basis for a fast clinical translation of these particles.

There is significant interest in in vivo CAR cell therapies, in particular, in the cancer immunotherapy market. The presently disclosed methods and particles possesses several advantages over the current state of the art. For example, the presently disclosed KNP delivery vehicle allows the pursuit of a simplified manufacturing and regulatory process, as it does not involve a cultured cell product and does not utilize targeting ligands. From a regulatory standpoint, the presently disclosed system is considered a nanoparticle product instead of a cell therapeutic. This characteristic allows the inconsistent phenotype and subsequent QC associated with ex vivo generated cell products to be avoided and offers a straightforward approval path during the commercialization process.

Further, manufacturing time is a fraction of that required for cultured cells, with a high degree of scalability for pilot plant and commercial production downstream. Notably, a clinical need to translate CAR cell therapies to solid tumors is present—in 2021 lung/bronchus, colorectal, pancreatic, and breast cancer accounted for nearly 50% of new cancer cases and deaths in the United States alone. National Cancer Institute, 2022b. CAR-Ms can infiltrate and kill a host of solid tumors, giving them a distinct advantage over competing CAR-T cell and NK systems. The introduction of CAR-Ms to this field allows us to tap into the broader cancer immunotherapy market, which saw projected global growth from $45.5B in 2016 to $117.1B in 2022. Mikulic, 2016. The avoidance of ex vivo manipulation and lower cost of goods in the presently disclosed system addresses the prohibitive high costs associated with current CAR cell therapies on the market and allows the biotechnology to have much broader accessibility to impact the world. Despite Medicare covering CAR-T cell therapies for certain non-Hodgkin lymphomas and B-cell precursor acute lymphoblastic leukemia, many private insurance plans do not provide this coverage. The wholesale cost of acquisition alone for Kymriah® or Yescarta® is $475,000 and $373,000, respectively, with the total cost for patients averaging over $700,000. Sahli et al., 2021. Modelling approaches estimate the cost of consumables and quality control in a GMP facility for CAR-T cell manufacturing to account for 75% of the total cost of goods sold. As such, the presently disclosed KNP technology has the commercial potential for adaptation as a mainstream cancer therapeutic by: (a) utilizing a simple high-throughput, scalable manufacturing system, (b) being used as an off-the-shelf treatment modality for a broad portfolio of solid tumors with enhanced efficacy, and (c) democratizing CAR immunotherapies by a drastic reduction in cost and complexity.

### Example 3

### Use of Multivalent Ions in Growth Buffer

### 3.1 Experimental

Luciferase mRNA (TriLink L-7202) was dissolved in 25 mM acetate buffer at a pH of 5.0. PBAE stock solution was diluted by a mixture of 90% vol % ethanol and 10% vol % the same acetate buffer to protonate amine groups. The PBAE-to-mRNA mass ratio was 30:1. The mRNA aqueous solution and the PBAE alcoholic solution were subjected to turbulent mixing by a T junction (IDEX Health and Science P-890) at a flow rate of 3 and 1 mL/min, respectively, controlled by two syringe pumps. This mixing step formed stable 100 nm mRNA-PBAE complex nanoparticles, which were subjected to dialysis against 1 mM acetate buffer at pH 5.0 to remove ethanol and excessive salt. These mRNA-PBAE nanoparticles were then mixed at 1:1 volume ratio with a solution of 75 mM NaCl containing 5 mM sodium citrate (final concentration of 37.5 mM NaCl and 2.5 mM sodium citrate), with its pH pre-adjusted to 7.0. Immediately after this mixing step (time=0 min), the mixture was aliquoted into a sizing cuvette onto a dynamic light scattering (DLS), and subjected to consecutive size measurements. Immediately after a last size measurement at around 7 min, an alcoholic solution of DMG-PEG at a concentration of 27 mg/mL was added into the mRNA-PBAE particles at a DMG-PEG-to-PBAE weight ratio of 1:10. Following a vortex for 20 s and an incubation period, another DLS measurement was carried out to confirm stabilization of the mRNA-PBAE particles.

### 3.2 Results

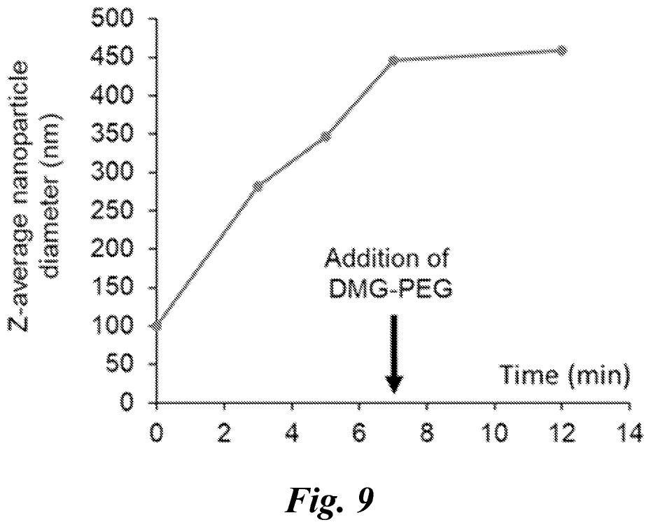

In FIG. 2, it takes mRNA-PBAE nanoparticles around 4 min to grow to a z-average particle size of 400 nm. The growth condition used in the experiment was 1× PBS containing 140 mM NaCl. Solely with nearly one fourth of this NaCl salt concentration (i.e., 37.5 mM), size growth could not happen. Addition of 2.5 mM sodium citrate, however, successfully induced size growth (FIG. 9), and the rate (around 6 min to 400 nm) was comparable to that in FIG. 2. This observation suggests that multivalent ions (citrate in this example) are effective in inducing mRNA-PBAE particle assembly and are presumably more effective than monovalent ions (chlorides), that they compensate the decreased concentration of NaCl. The presence of such multivalent ions did not impact subsequent stabilization by DMG-PEG. This finding expands the engineering space of the buffer that can be used to induce size growth of mRNA-PBAE nanoparticles.

### Example 4

### Application of the Nanoparticle System for Gene Editing Purposes

### 4.1 Experimental

The mRNA-PBAE particles at 200, 400, and 900 nm were prepared as described hereinabove in Example 3 and FIG. 9, except that the mRNA solution is replaced with a mixture of mCherry mRNA (TriLink L-7203), CRISPR Cas9 mRNA (L-7206) and guide RNA (gRNA) against eGFP (in-house designed and synthesized by IVT) at weight ratio of 2:2:1. Stable GFP-expressing B16F10 cells were seeded in 24-well plate at a density of 50000 cells/well, 24 h prior to experiment. mRNA-PBAE particles were dosed into the cells at a final mRNA concentration of 1 μg/mL. After 4 h, the particle-containing medium was removed and refreshed with particle-free full culture medium. The cells were continued to be cultured for an additional 20 h, before being detached by trypsin-EDTA, suspended in PBS containing 5% FBS and analyzed by flow cytometry.

### 4.2 Results

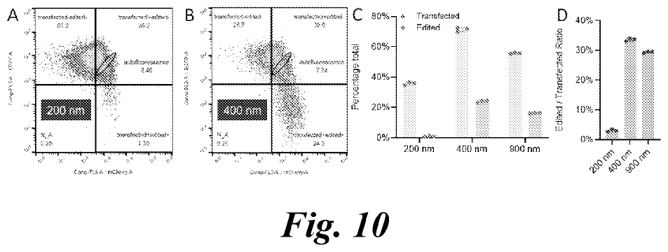

With the payload mixture, successfully transfected cells would have mCherry expression (from the mCherry mRNA cargo), while successfully edited cells would have decreased eGFP expression intensity (from the Cas9 mRNA and eGFP gRNA cargos). FIG. 10A showed that 200-nm particles resulted in around 35% cells transfected, however, only 1.3% cells were edited. FIG. 10B showed that 400-nm particles resulted in around 70% cells transfected (top-right and bottom-right quadrants), significantly higher than that of 200-nm particles. Besides, out of these 70% cells, nearly one third (i.e., 24.5% of all cells) were successfully edited. FIG. 10C summarizes the particle size effect and showed that 900-nm particles resulted in similar outcomes as those seen from 400-nm particles; while FIG. 10D summarizes the relative efficiency of successful editing to successful transfection.

Successful transfection requires successful intracellular delivery of only one mRNA (i.e., mCherry mRNA), however, successful gene editing requires successful intracellular delivery of both the Cas9 mRNA and gRNA with a large quantity. As shown FIG. 2E, 400-nm or 900-nm particles have significantly higher payload capacity (reflected as mRNA payload) than 200-nm particles. Such greatly increased payload enabled (1) encapsulation of all three cargos with substantial quantity in the same particle; (2) intracellular delivery of all three cargos efficiently. This shows a unique advantage of the size engineering of mRNA-PABE particles described herein.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Waldman A D, Fritz J M, Lenardo M J, A guide to cancer immunotherapy: from T cell basic science to clinical practice, Nature Reviews Immunology, 20 (11): 651-668 (2020).
National Cancer Institute, “CAR T cells: Engineering immune cells to treat cancer,” https://www.cancer.gov/about-cancer/treatment/research/car-t-cells [Accessed March 2022].
Patel S, et al., Beyond CAR T cells: Other cell-based immunotherapeutic strategies against cancer, Frontiers in Oncology, 9:196 (2019).
Parayath N N, Stephan M T, In situ programming of CAR T cells, Annual Review of Biomedical Engineering, 23:385-405 (2021).
Tumeh P C, et al, The impact of ex vivo clinical grade activation protocols on human T-cell phenotype and function for the generation of genetically modified cells for adoptive cell transfer therapy,” Journal of Immunotherapy, vol. 33 (8): 759-768 (2010).
National Cancer Institute, Surveillance, Epidemiology, and End Results Program, Cancer stat facts: Common cancer sites, http://seer.cancer.gov/statfacts/html/common.html [Accessed March 2022].
Kang M, et al, Nanocomplex-mediated in vivo programming to chimeric antigen receptor-M1 macrophages for cancer therapy, Advanced Materials, 33 (43): e2103258 (2021).
Karlsson J, Rhodes K R, Green J J, Tzeng S Y, Poly(beta-amino ester) s as gene delivery vehicles: challenges and opportunities, Expert Opinion on Drug Delivery, 17:1395-1310 (2020).
Mikulic M, Global cancer immunotherapy market in 2016 and projection for 2022, Statista, 2016.
Sahli B, Eckwright D, Darling E, Gleason P P, Leach J W, Chimeric antigen receptor t-cell (CAR-T) therapy real-world assessment of total cost of care and clinical events for the treatment of relapsed or refractory lymphoma among 15 million commercially insured members, Academy of Managed Care Pharmacy (AMCP) Virtual Annual Meeting, (2021).

Although the foregoing subject matter has been described in some detail by way of illustration and example for purposes of clarity of understanding, it will be understood by those skilled in the art that certain changes and modifications can be practiced within the scope of the appended claims.

1. A method for preparing a plurality of hybrid nanoparticles having a defined size, the method comprising:
(a) mixing a biodegradable cationic polymer and one or more nucleic acids to form a first plurality of charge-neutralized complexes;
(b) disposing the plurality of charge-neutralized complexes in a buffer solution for a period of time to induce particle growth to form a second plurality of charge-neutralized complexes, wherein the second plurality of charged neutralized complexes has a particle size greater than a particle size of the first plurality charge-neutralized complexes; and
(c) adding a hydrophobic PEGylated lipid to the second plurality of charge-neutralized complexes to quench particle growth and to form a plurality of hybrid nanoparticles having a defined size.

2. The method of claim 1, wherein:
(a) the first plurality of charge-neutralized complexes are formed under low salt concentration and low pH conditions, wherein the low salt concentration ranges from an ionic strength equivalent to about 1 mM to about 300 mM NaCl and the high pH ranges from about 2 to about 9.0; and/or
(b) the second plurality of charge-neutralized complexes are formed under high salt concentration and high pH conditions in the presence of one or more multivalent ions.

3. (canceled)

4. The method of claim 2, wherein the one or more multivalent ions comprise a negatively-charged ion selected from phosphate, citrate, EDTA, pyrophosphate, ATP, tripolyphosphate, and hexametaphosphate or a positively-charged magnesium, calcium, ferrous, and aluminum.

5.-6. (canceled)

7. The method of claim 1, wherein the biodegradable cationic polymer and the one or more nucleic acids are mixed through pipetting, at a T junction flow path, in a microfluidic channel or mixer, in a multi-inlet vortex mixer, or in a confined impinging jet (CIJ) mixer.

8.-9. (canceled)

10. The method of claim 1, wherein:
(a) the biodegradable cationic polymer and the one or more nucleic acids are mixed at a ratio of about 3:1 nucleic acid:cationic polymer;
(b) the buffered solution comprises phosphate buffered saline (PBS); and/or
(c) the period of time the first plurality of charge-neutralized complexes is disposed in the buffer solution has a range from about 0.1 min to about 300 min.

11.-13. (canceled)

14. The method of claim 1, wherein the PEGylated lipid comprises 1,2-dimyristoyl-sn-glycero-3-methoxypolyethylene glycol (DMG-PEG).

15. The method of claim 14, wherein:
(a) the PEGylated lipid is DMG-PEG2000;
(b) the PEGylated lipid is DMG-PEG2000 and the DMG-PEG2000 comprises greater than about 5% of a mass concentration of the hybrid nanoparticle; and/or
(c) the PEGylated lipid is DMG-PEG2000 and the DMG-PEG2000 comprises between about 5% to about 20% of the mass concentration of the hybrid nanoparticle.

16.-17. (canceled)

18. The method of claim 1, wherein the PEGylated lipid further comprises a chemically-active moiety, wherein the chemically-active moiety can be functionalized with a target ligand or other biologically active chemical structures.

19. (canceled)

20. The method of claim 1, wherein the one or more nucleic acids is selected from an antisense oligonucleotide, cDNA, genomic DNA, guide RNA, plasmid DNA (pDNA), including a mixture of different species of pDNA, vector DNA, mRNA, miRNA, piRNA, shRNA, and siRNA.

21. (canceled)

22. The method of claim 20, wherein the plurality of hybrid nanoparticles comprises between about 2 to about 1500 copies of mRNA per particle.

23. The method of claim 1, wherein the plurality of hybrid nanoparticles have:
(a) an average particle size having a range from about 50 nm to about 1000 nm; about 50 nm to about 900 nm; about 50 nm to about 800 nm; about 50 nm to about 700 nm; about 50 nm to about 600 nm; about 50 nm to about 500 nm; about 50 nm to about 400 nm; about 50 nm to about 300 nm; about 50 nm to about 200 nm; and about 50 nm to about 100 nm;
(b) a zeta-potential of between about 2 mV and about 6 mV; and/or
(c) an encapsulation efficiency of between about 80% to about 100%.

24.-26. (canceled)

27. The method of claim 1, wherein the biodegradable cationic polymer comprises a poly(beta-amino ester) (PBAE).

28. The method of claim 27, wherein the PBAE comprises a compound of formula (I): 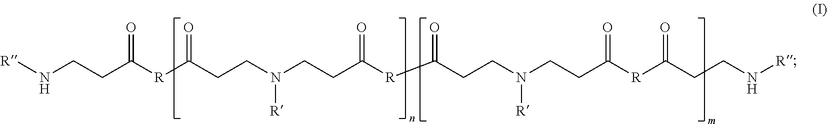
wherein:
m and n are each independently an integer from 1 to 10,000;
(a) R is selected from: 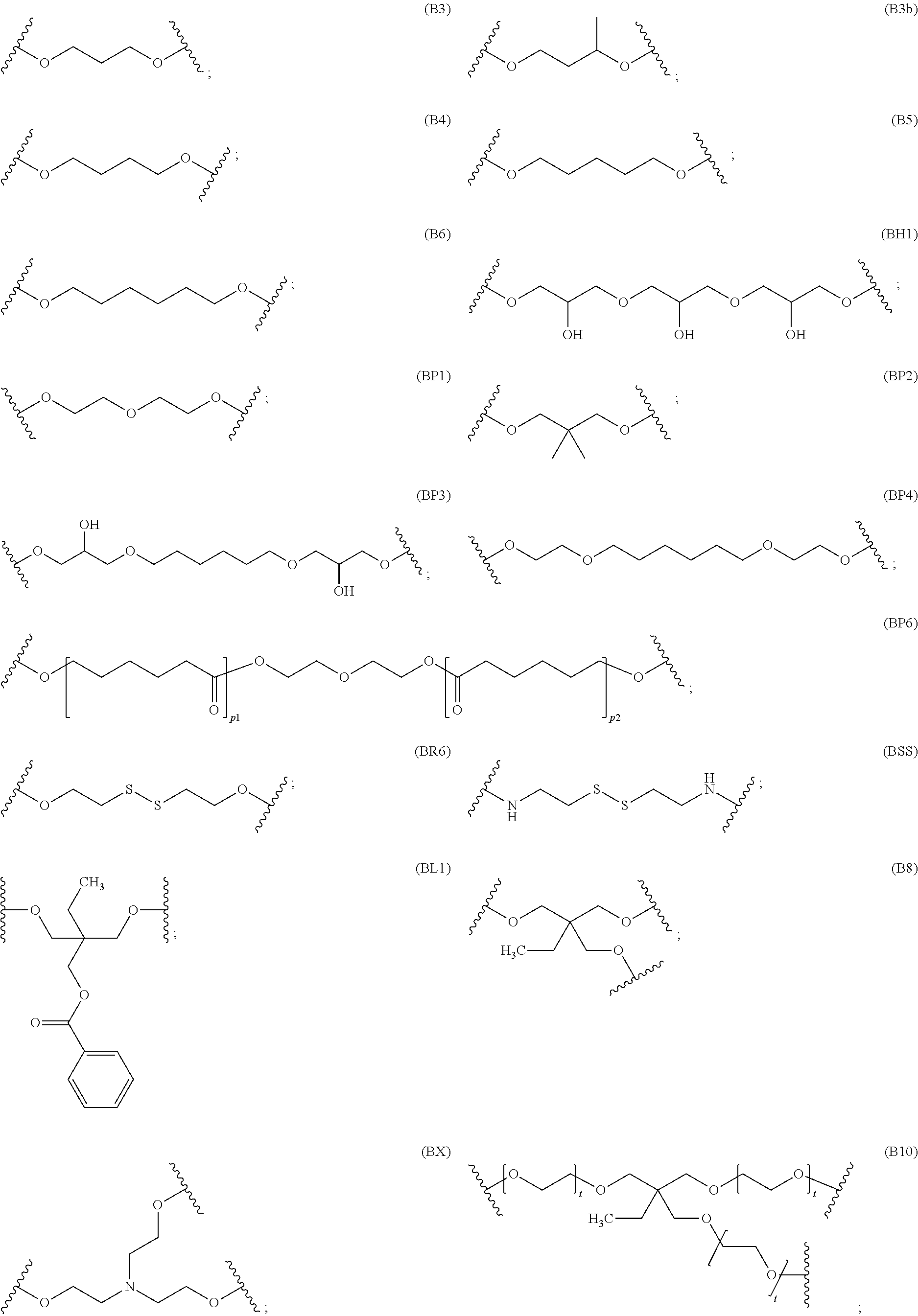 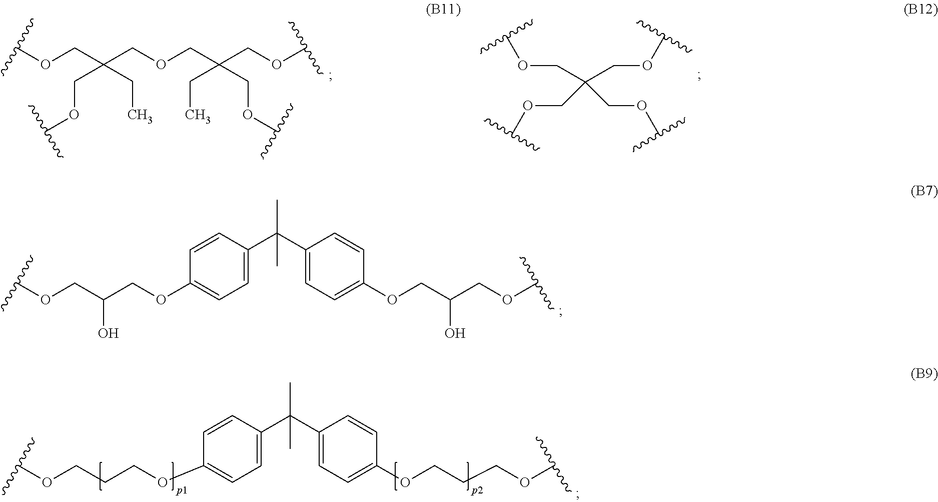
wherein each p1, p2, and t is independently an integer selected from the group consisting of 1, 2, 3, 4, 5, 6, 7, 8, 9, and 10;
(b) at least one R′ comprises: 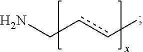
wherein x is an integer selected from 0, 1, 2, 3, 4, 5, 6, 7, 8, 9, 10, 11, 12, 13, 14, 15, 16, 17, 18, 19, and 20; and wherein 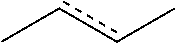
can De a single or double bond in one or more x repeating units; or at least one R′ is a hydrophilic sidechain selected from: 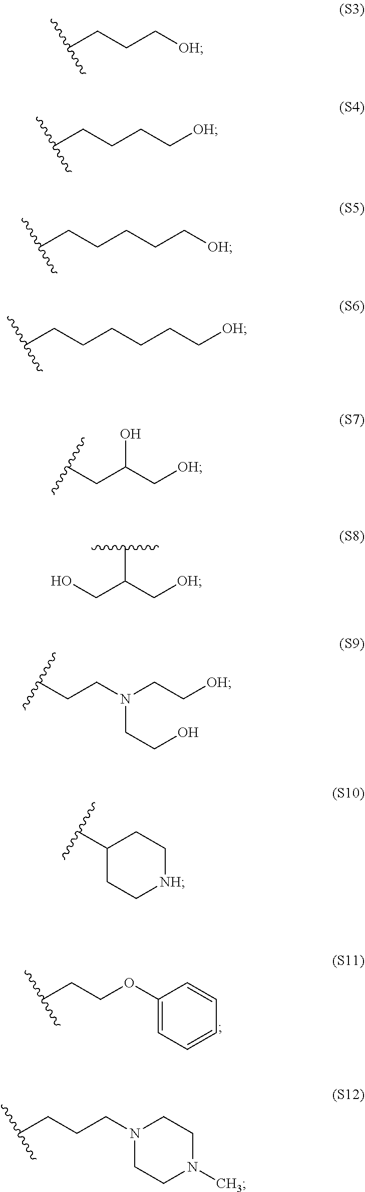 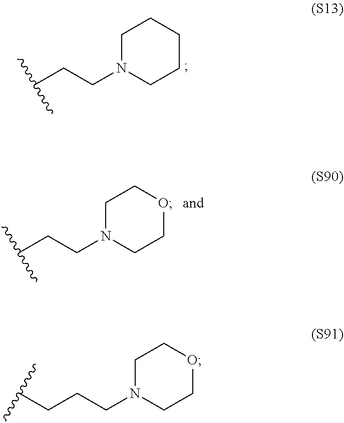
and
(c) R″ is selected from the group consisting of: 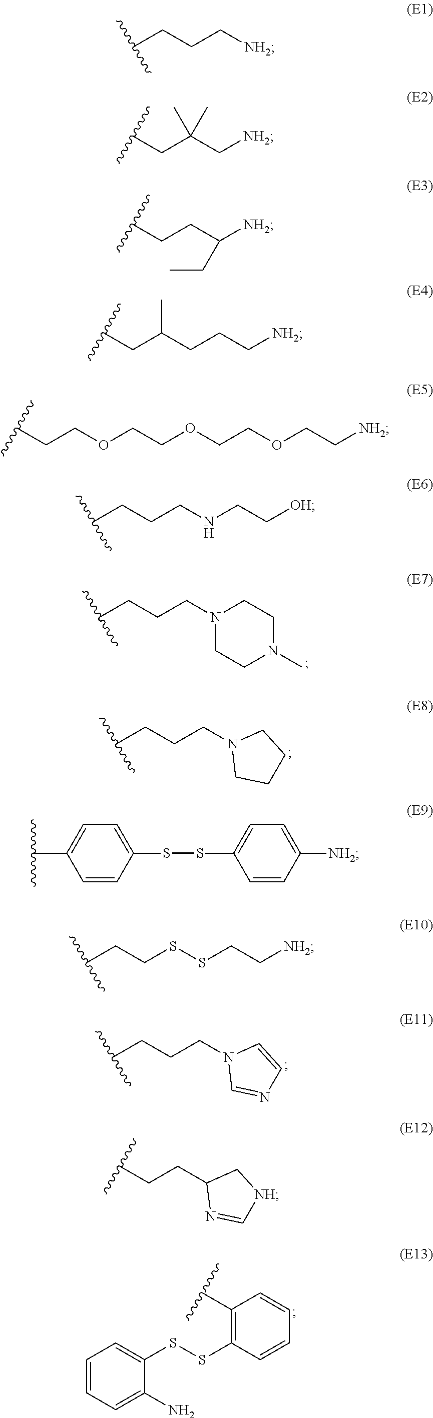 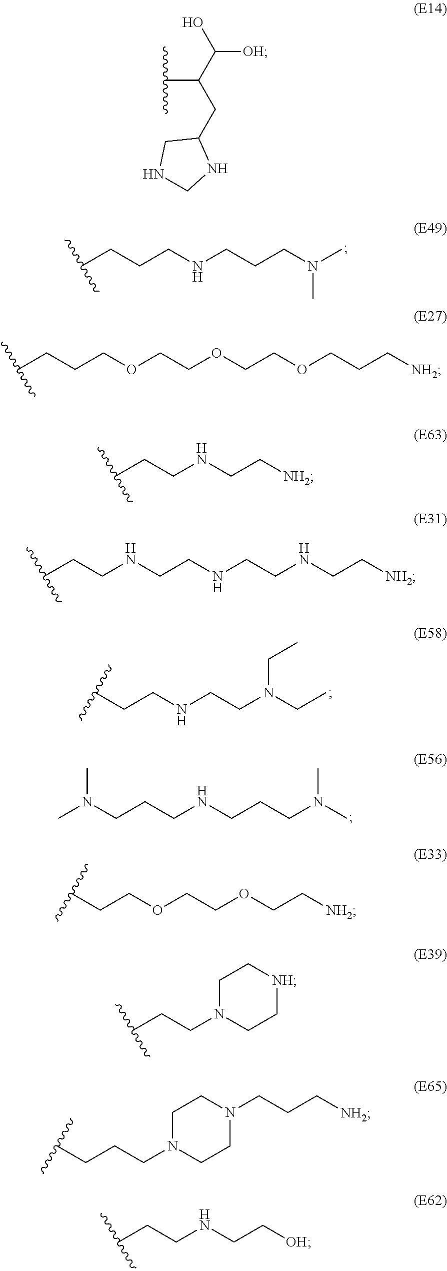 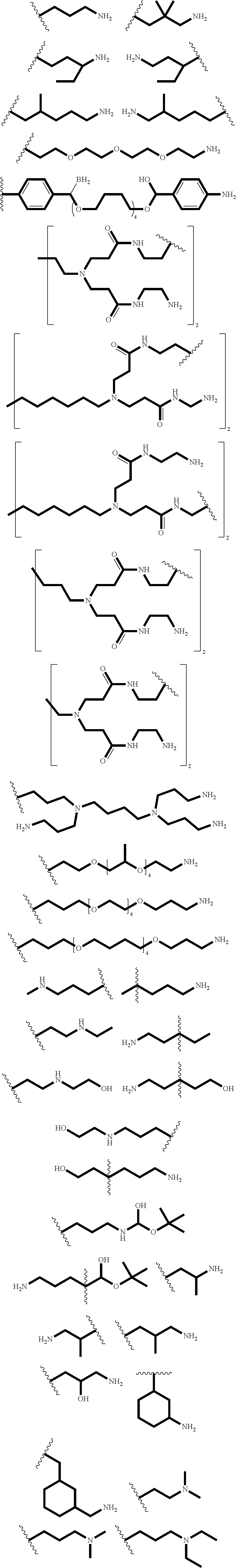 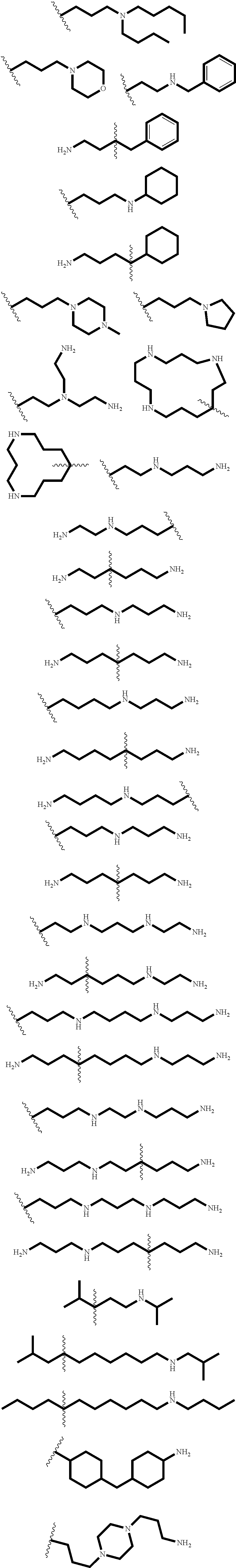 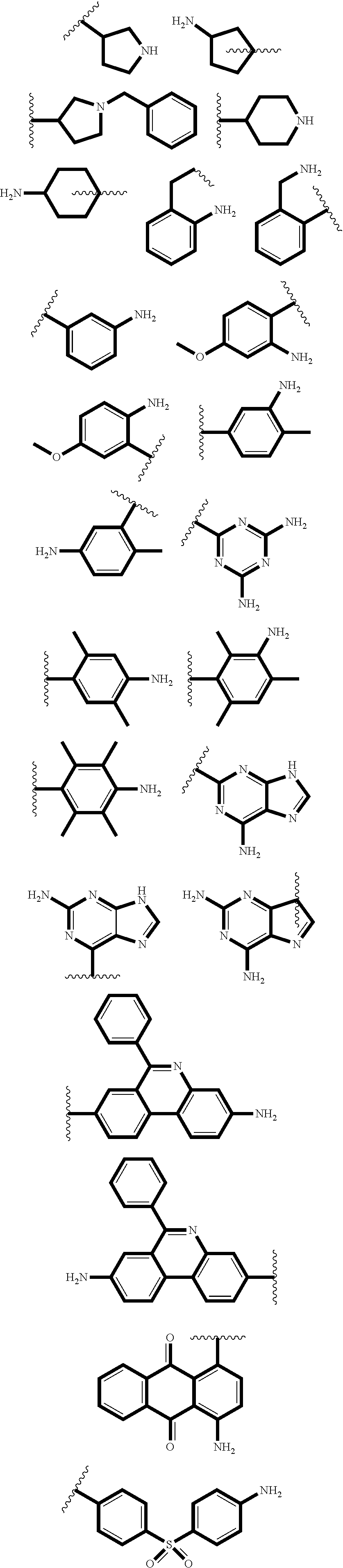 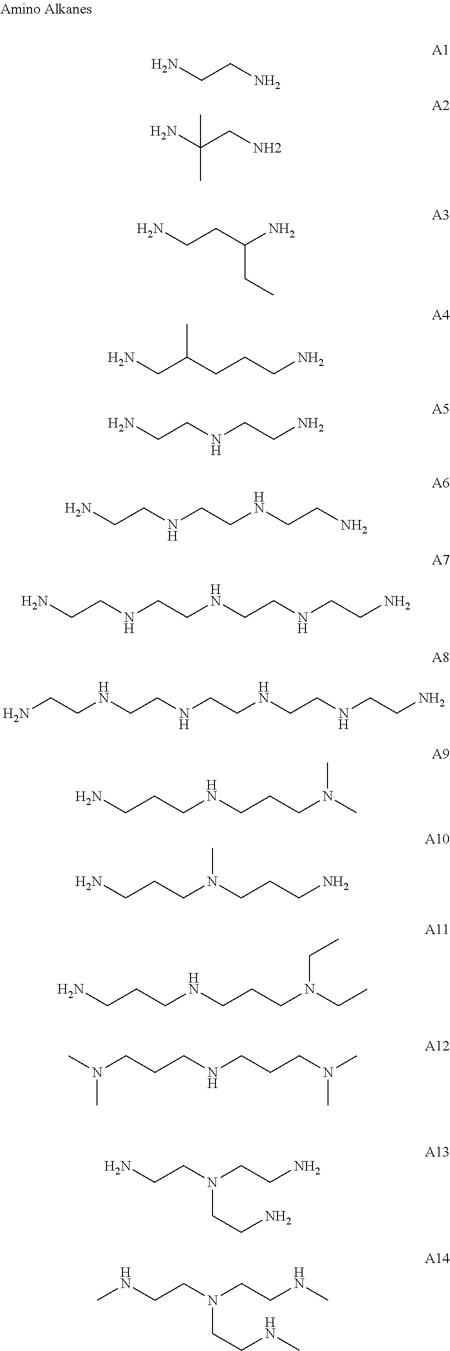 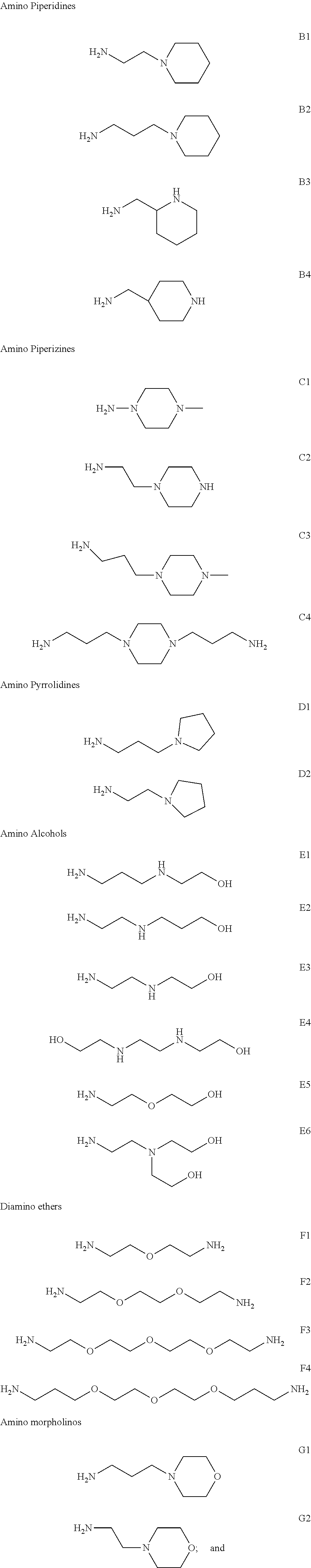 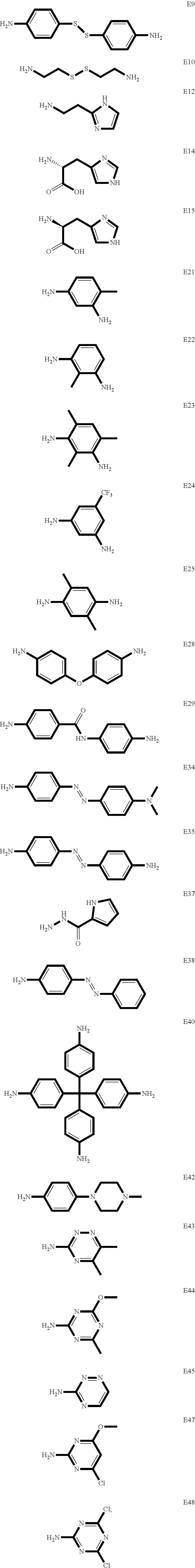
and pharmaceutically ble salts thereof.

29.-31. (canceled)

32. The method of claim 28, wherein at least one R′ is selected from:

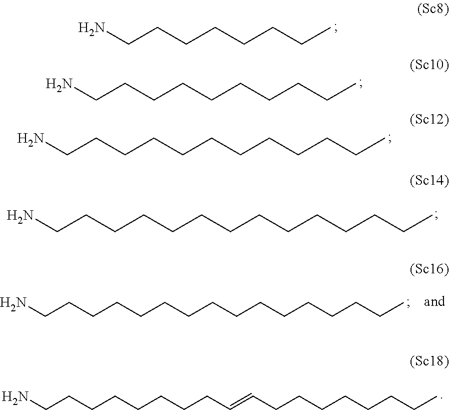

33.-36. (canceled)

37. The method of claim 28, wherein the compound of formula (I) is:

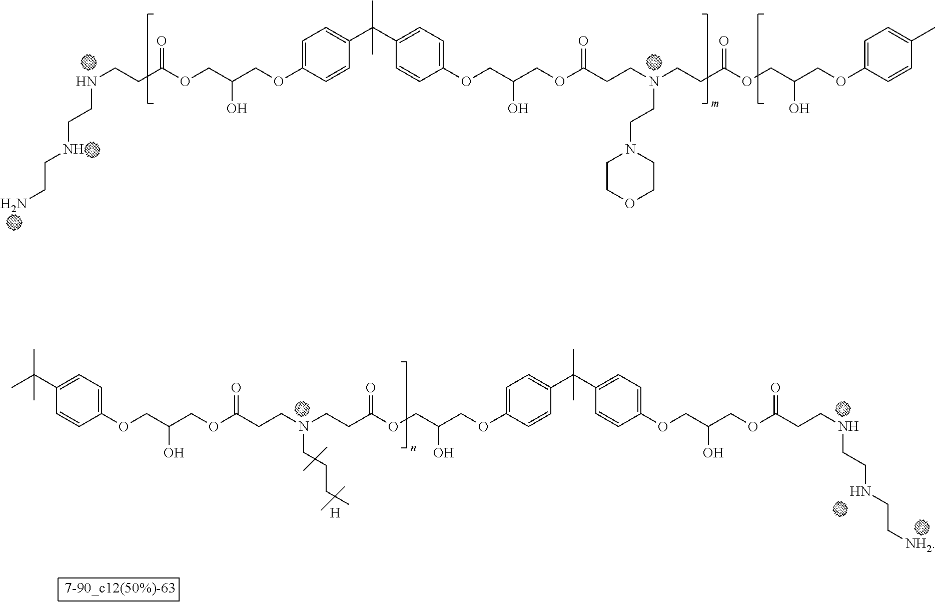

38. The method of claim 1, further comprising an excipient for cryo-preservation of the plurality of particles, wherein the excipient is selected from a saccharide and a sugar alcohol.

39. (canceled)

40. The method of claim 38, wherein the saccharide is selected from a monosaccharide, a disaccharide, and a polysaccharide and the sugar alcohol is selected from arabinose, glucose, fructose, ribose, mannose, sucrose, trehalose, lactose, maltose, a starch, dextran, mannitol, and sorbitol.

41. (canceled)

42. The method of claim 40, wherein the sugar alcohol is 15% w/v trehalose.

43. A hybrid nanoparticle comprising a PBAE, a nucleic acid, and a PEGylated lipid, wherein nucleic acid comprises mRNA, the PEGylated lipid comprises DMG-PEG2000, and the PBAE comprises a compound of the following formula:

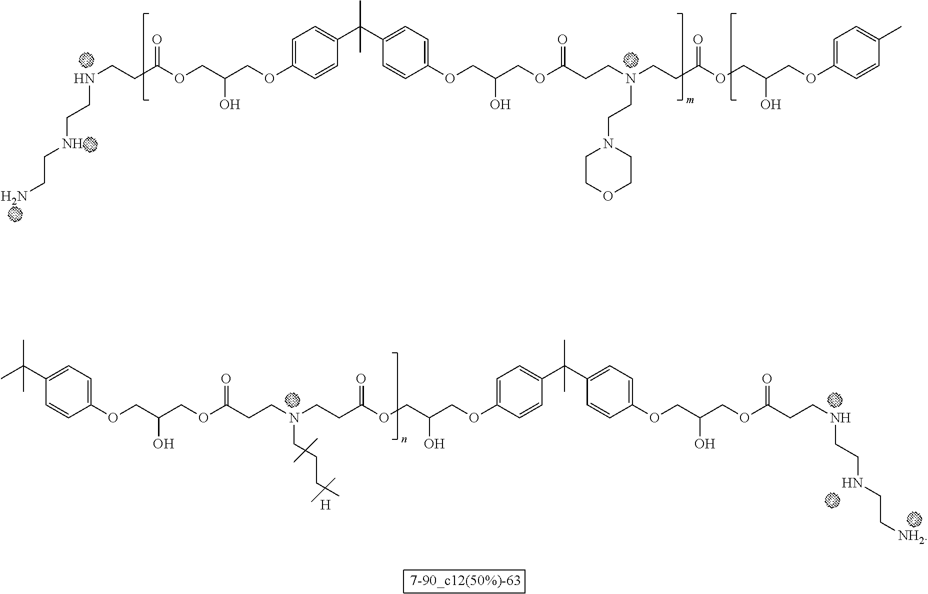

44. The hybrid nanoparticle of claim 43, wherein:
(a) the hybrid nanoparticle has particle size of about 400 nm;
(b) the DMG-PEG2000 comprises between about 5% to about 20% of a mass concentration of the nanoparticle;
(c) the plurality of hybrid nanoparticles have between about 2 to about 1500 copies of mRNA per particle;
(d) the plurality of hybrid nanoparticles have a zeta-potential of between about 2 mV and about 6 mV;
(e) the hybrid nanoparticle has an encapsulation efficiency of between about 80% to about 100%; and/or
(f) the PEGylated lipid further comprises a chemically-active moiety, wherein the chemically-active moiety can be functionalized with a targeting ligand or other biologically active chemical structures.

45.-50. (canceled)

51. A method for transfecting a cell, delivering mRNA to a tissue, delivering a gene to a subject, and/or treating a disease, condition, or disorder, the method comprising administering to the cell, tissue, or subject a hybrid nanoparticle of claim 43.

52. The method of claim 51, comprising ex vivo or in vitro transfection and gene editing.

53. (canceled)

54. The method of claim 51, wherein the tissue is selected from lung, liver, kidney, heart, and spleen.

55. (canceled)

56. The method of claim 51, wherein the administration is in vivo.

57. The method of claim 56, comprising intravenous administration.

58. (canceled)

59. The method of claim 51, wherein the disease, condition, or disorder comprises cancer.
